(19) United States
(12) Patent Application Publication
(10) Pub. No.: US 2026/0020870 A1
(43) Pub. Date: Jan. 22, 2026

(54) INSERT CATHETER WITH SIDE PORT

(71) Applicant: Imperative Care, Inc., Campbell, CA (US)

(72) Inventors: Mathurin Boutte, San Jose, CA (US); Craig Mar, Fremont, CA (US); Ashoor Shahbazi Yourgenlow, San Jose, CA (US)

(21) Appl. No.: 19/271,560

(22) Filed: Jul. 16, 2025

Related U.S. Application Data
(60) Provisional application No. 63/673,631, filed on Jul. 19, 2024.

(51) Int. Cl. A61B 17/22; A61M 25/00; A61M 25/06

(52) U.S. Cl. CPC A61B 17/22031; A61M 25/0026; A61M 25/0068; A61M 25/0662; A61B 2017/22038; A61M 2210/127

(57) ABSTRACT

An insert catheter having an elongate flexible body. The elongate flexible body has a proximal end, a distal end, a length of at least about 160 cm, a lumen extending from the proximal end to the distal end, and a side port in fluid communication with the lumen. The side port is positioned between about 30 cm to about 105 cm from the distal end of the elongate flexible body. A guidewire can extend through the lumen and the side port. The side port is configured to be positioned within a second interventional device during a neurovascular procedure. The insert catheter can have a pre-shaped tip on a distal portion of the elongate flexible body. The insert catheter can have a rail extending from the side port to the proximal end of the elongate flexible body. The rial can be configured to receive the guidewire.

### INCORPORATION BY REFERENCE TO ANY PRIORITY APPLICATIONS

Any and all applications for which a foreign or domestic priority claim is identified in the application data sheet as filed with the present application are hereby incorporated by reference under 37 C.F.R. § 1.57. The present application claims priority to U.S. Provisional Patent Application No. 63/673,631, filed Jul. 19, 2024, titled INSERT CATHETER WITH SIDE PORT, the entire content of which is incorporated by reference herein for all purposes and forms as part of this specification.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to a rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stoke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mm Hg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Obstructive emboli have also been mechanically removed from various sites in the vasculature for years. Mechanical therapies have involved capturing and removing the clot, dissolving the clot, disrupting and suctioning the clot, and/or creating a flow channel through the clot. One of the first mechanical devices developed for stroke treatment is the MERCI Retriever System (Concentric Medical, Redwood City, Calif.). A balloon-tipped guide catheter is used to access the internal carotid artery (ICA) from the femoral artery. A microcatheter is placed through the guide catheter and used to deliver the coil-tipped retriever across the clot and is then pulled back to deploy the retriever around the clot. The microcatheter and retriever are then pulled back, with the goal of pulling the clot, into the balloon guide catheter while the balloon is inflated and a syringe is connected to the balloon guide catheter to aspirate the guide catheter during clot retrieval. This device has had initially positive results as compared to thrombolytic therapy alone.

Other thrombectomy devices utilize expandable cages, baskets, or snares to capture and retrieve clot. Temporary stents, sometimes referred to as stentrievers or revascularization devices, are utilized to remove or retrieve clot as well as restore flow to the vessel. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Notwithstanding the foregoing, there remains a need for new devices and methods for treating vasculature occlusions in the body, including acute ischemic stroke and occlusive cerebrovascular disease. In particular, as will be discussed in more detail below, because of the variation in levels of tortuosity and the large variability of certain segments of the intracranial carotid artery (e.g., the petrous-cavernous path) in stroke patients, there is a need for an anatomy matched catheter design.

### SUMMARY

There is provided in accordance with one aspect of the present disclosure a system for a neurovascular procedure. The system can include an introducer sheath having a central lumen; a first interventional device having a first lumen and configured to extend along the central lumen of the introducer sheath; a second interventional device having a second lumen and configured to extend along the first lumen of the first interventional device; and a third interventional device configured to extend along the second lumen of the second interventional device. The third interventional device can include an elongate flexible body having a proximal end and a distal end. The elongate flexible body having a length of at least about 160 cm. The third interventional device can include a third lumen extending from the proximal end to the distal end, and a side port in fluid communication with the third lumen and positioned along the elongate flexible body. The side port can be positioned between about 30 cm to about 105 cm from the distal end of the elongate flexible body. The system can include a guidewire configured to extend through the third lumen and the side port. When a distal end of the first interventional device or a distal end of the second interventional device are positioned beyond the distal end of the elongate flexible body of the third interventional device, a distance between the introducer sheath and a portion of the guidewire located immediately proximal to the second interventional device ranges from about 20 cm to about 40 cm.

In some aspects, the elongate flexible body can include a pre-shaped tip on a distal portion of the elongate flexible body. The pre-shaped tip of the elongate flexible body can include a first curve defining a first concave side and a first convex side; a second curve defining a second concave side and a second convex side; and a third curve defining a third concave side and a third convex side. A longitudinal axis of the elongate flexible body and a first section of the pre-shaped tip distal to the first curve can form an angle from about 120° to about 180°. In some cases, the first section of the pre-shaped tip and a second section of the pre-shaped tip distal to the second curve can form an angle from about 5° to about 65°. The longitudinal axis of the elongate flexible body and a third section of the pre-shaped tip distal to the third curve can form an angle from about 65° to about 125°. In some cases, a distance between the distal end of the elongate flexible body and a longitudinal axis of the elongate flexible body along an axis perpendicular to the longitudinal axis is between about 1 mm and about 30 mm. In some aspects, a distance between a lateral-most point of the pre-shaped tip and a longitudinal axis of the elongate flexible body along an axis perpendicular to the longitudinal axis is between about 5 mm and about 35 mm.

In some aspects, the pre-shaped tip of the elongate flexible body includes a first curve defining a first concave side and a first convex side; a second curve defining a second concave side and a second convex side; and a third curve defining a third concave side and a third convex side. A longitudinal axis of the elongate flexible body and a first section of the pre-shaped tip distal to the first curve can form an angle from about 60° to about 180°. In some cases, the first section of the pre-shaped tip and a second section of the pre-shaped tip distal to the second curve can form an angle from about 5° to about 30°. The longitudinal axis of the elongate flexible body and a third section of the pre-shaped tip distal to the third curve can form an angle from about 45° to about 135°. In some cases, a distance between the distal end of the elongate flexible body and a longitudinal axis of the elongate flexible body along an axis perpendicular to the longitudinal axis can be between about 2 mm and about 22 mm. A distance between the distal end of the elongate flexible body and a lateral-most point of the pre-shaped tip along an axis perpendicular to a longitudinal axis of the elongate flexible body can be between about 15 mm and about 75 mm.

In some aspects, the elongate flexible body includes a rail extending from the side port to the proximal end of the elongate flexible body.

There is also provided in accordance with one aspect of the present

disclosure an insert catheter. The insert catheter can include an elongate flexible body having a proximal end and a distal end, the elongate flexible body having a length of at least about 160 cm, a lumen extending from the proximal end to the distal end; and a side port in fluid communication with the lumen and positioned along the elongate flexible body. The side port can be positioned between about 30 cm to about 105 cm from the distal end of the elongate flexible body. The side port can be configured to receive a guidewire and place the guidewire in communication with the lumen. The side port can be configured to be positioned within a second interventional device during a neurovascular procedure.

In some aspects, the insert catheter includes a pre-shaped tip on a distal portion of the elongate flexible body. The pre-shaped tip can include a first curve defining a first concave side and a first convex side; a second curve defining a second concave side and a second convex side; and a third curve defining a third concave side and a third convex side. A longitudinal axis of the elongate flexible body and a first section of the pre-shaped tip distal to the first curve can form an angle from about 120° to about 180°. The first section of the pre-shaped tip and a second section of the pre-shaped tip distal to the second curve can form an angle from about 5° to about 65°. The second section of the pre-shaped tip and a third section of the pre-shaped tip distal to the third curve can form an angle from about 65° to about 125°. In some cases, a distance between the distal end of the elongate flexible body and a longitudinal axis of the elongate flexible body along an axis perpendicular to the longitudinal axis is between about 1 mm and about 30 mm. In some aspects, a distance between a lateral-most point of the pre-shaped tip and a longitudinal axis of the elongate flexible body along an axis perpendicular to the longitudinal axis is between about 5 mm and about 35 mm.

In some aspects, the pre-shaped tip of the elongate flexible body can include a first curve defining a first concave side and a first convex side; a second curve defining a second concave side and a second convex side; and a third curve defining a third concave side and a third convex side. A longitudinal axis of the elongate flexible body and a first section of the pre-shaped tip distal to the first curve can form an angle from about 60° to about 180°. The first section of the pre-shaped tip and a second section of the pre-shaped tip distal to the second curve can form an angle from about 5° to about 30°. In some cases, the second section of the pre-shaped tip and a third section of the pre-shaped tip distal to the third curve can form an angle from about 45° to about 135°. A distance between the distal end of the elongate flexible body and a longitudinal axis of the elongate flexible body along an axis perpendicular to the longitudinal axis is between about 2 mm and about 22 mm. A distance between the distal end of the elongate flexible body and a lateral-most point of the pre-shaped tip along an axis perpendicular to a longitudinal axis of the elongate flexible body is between about 15 mm and about 75 mm.

In some aspects, the insert catheter includes a rail extending from the side port to the proximal end of the elongate flexible body.

There is also provided in accordance with one aspect of the present disclosure, a method of gaining and maintaining access to an ostium of the aortic arch using a stack of interventional devices, the stack of interventional devices including a first interventional device, a second interventional device, and a third interventional device. The method can include placing an introducer sheath into an artery of a patient; positioning the second interventional device inside the first interventional device; and positioning the third interventional device inside the second interventional device such that a side port of the third interventional device is encapsulated by the second interventional device. The method can also include introducing the stack of interventional devices into the vasculature of the patient via the introducer sheath; with a distal end of the third interventional device positioned beyond a distal end of the first interventional device and a distal end of the second interventional device, advancing the stack of interventional devices through the vasculature of the patient until a distal end of the third interventional device is positioned within the aortic arch; and, with the distal end of the third interventional device positioned inside the aortic arch, adjusting a position of the distal end of the third interventional device so the distal end of the third interventional device is positioned within an ostium of the aortic arch. The method can also include, with the distal end of the third interventional device positioned in the ostium of the aortic arch, advancing at least one of a guidewire, the first interventional device, or the second interventional device beyond the distal end of the third interventional device to get access to supra-aortic vessels. The side port can be positioned between a proximal end of the third interventional device and the distal end of the third interventional device. The proximal end of the second interventional device can include a hub. The guidewire can extend through a lumen of the third interventional device. In some cases, the guidewire can exit the third interventional device via the side port. A portion of the guidewire positioned outside of the stack of interventional devices can be first available immediately proximal to the hub of the second interventional device.

In some aspects, a distance between a proximal end of the introducer sheath and the hub of the second interventional device can be between about 20 cm to about 40 cm, when the stack is positioned inside the vasculature of the patient.

There is also provided in accordance with one aspect of the present disclosure, a stack of interventional devices for an interventional procedure. The stack can include a first interventional device; a second interventional device including a hub and configured to be positioned within the first interventional device; and a third interventional device including an elongate body and a hub, wherein the third interventional device is configured to be positioned within the second interventional device. The elongate body can include a proximal end, wherein the hub is coupled to the proximal end of the elongate body; a distal end; a lumen extending from the proximal end to the distal end; and a side port positioned on the elongate body between the proximal end and the distal end such that a proximal portion of the elongate body extends from the proximal end to the side port, and a distal portion of the elongate flexible body extends from the distal end to the side port. The third interventional device can be configured to receive a guidewire via the side port. When the second interventional device is positioned inside the first interventional device and the third interventional device is positioned inside the second interventional device, the second interventional device, the third interventional device, and the guidewire can be configured to be manipulated using a single hand.

In some aspects, the elongate body can include a rail extending from the side port to the proximal end of the elongate body. The proximal portion of the elongate body can include a hypotube. In some cases, the side port can be positioned between about 30 cm to about 105 cm from the distal end of the third interventional device. The side port can be positioned inside the second interventional device when the distal end of the third interventional device is positioned beyond a distal end of the first interventional device or a distal end of the second interventional device. In some aspects, the stack includes an introducer sheath. The introducer sheath can be configured to receive the first interventional device, the second interventional device, and the third interventional device. In some cases, a distance between the introducer sheath and a portion of the guidewire located immediately proximal to the hub of the second interventional device ranges from about 20 cm to about 40 cm.

### BRIEF DESCRIPTION OF THE DRAWINGS

FIG. 1 illustrates a side elevational schematic view of an example of an intracranial aspiration catheter with a distal segment in a proximally retracted configuration.

FIG. 2 illustrates a side elevational view of the intracranial aspiration catheter of FIG. 1, with the distal segment in a distally extended configuration.

FIG. 3 illustrates cerebral arterial vasculature including the Circle of Willis, and an access catheter positioned at an occlusion in the left carotid siphon artery.

FIGS. 4A-4E show a sequence of steps involved in positioning of the catheter and aspirating obstructive material from the middle cerebral artery and the subsequent removal of the catheter following aspiration of the obstructive material.

FIGS. 5A-5F depict a sequence of steps to access a neurovascular occlusion for aspiration.

FIGS. 6A-6F depicts an alternative sequence of steps involved in accessing a neurovascular occlusion for aspiration.

FIG. 7A illustrates a cross-sectional view of a catheter according to an embodiment.

FIG. 7B illustrates the catheter shown in FIG. 7A with a rapid exchange port.

FIG. 7C illustrates a portion of a catheter according to one embodiment with a braid wrapped around an inner liner.

FIG. 8A illustrates a top view of an insert catheter with a side port along the tubular body of the insert catheter.

FIG. 8B illustrates a side view of the insert catheter shown in FIG. 8A.

FIG. 9A illustrates another side view of the insert catheter shown in FIGS. 8A-8B.

FIG. 9B illustrates a cross section of the insert catheter through the line B-B in FIG. 9A.

FIG. 9C illustrates a cross section of the insert catheter through the line C-C in FIG. 9A.

FIG. 9D illustrates Detail K shown in FIG. 9B.

FIG. 9E illustrates Detail J shown in FIG. 9C.

FIG. 10 illustrates an example of a pre-shaped tip for an insert catheter.

FIG. 11 illustrates another example of a pre-shaped tip for an insert catheter.

FIGS. 12A-12B illustrate an example of a stack of interventional devices including an insert catheter.

FIG. 12C schematically illustrates an example of manual control of the stack of interventional devices shown in FIGS. 12A-12B.

### DETAILED DESCRIPTION

### Overview

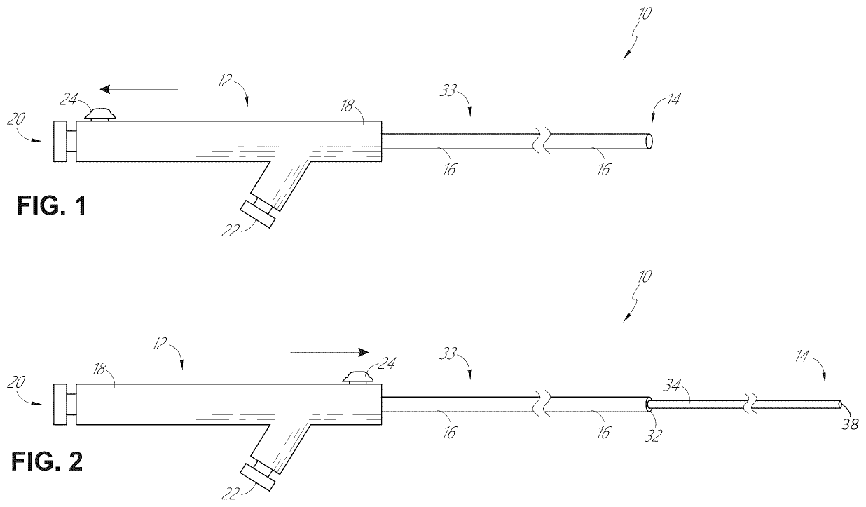

FIG. 1 illustrates an embodiment of a catheter 10 in accordance with one aspect of the present disclosure. Although primarily described in the context of an axially extendable distal segment aspiration catheter with a single central lumen, the presently disclosed catheter can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the presently disclosed catheter will be described primarily in the context of removing obstructive material from remote vasculature in the brain, but has applicability as an access catheter for delivery and removal of any of a variety of diagnostics or therapeutic devices with or without aspiration.

The catheters disclosed herein may readily be adapted for use throughout the body wherever it may be desirable to distally advance a low profile distal catheter segment from a larger diameter proximal segment. For example, the presently disclosed axially extendable catheter shafts may be dimensioned for use throughout the coronary and peripheral vasculature, the gastrointestinal tract, the urethra, ureters, Fallopian tubes and other lumens and potential lumens, as well. The telescoping structure presently disclosed may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants. Examples of such catheters are illustrated in, for example, U.S. Pat. No. 10,183,145 to Yang, et al., and U.S. Pat. No. 10,835,272 to Yang, et al. the disclosure of which are incorporated in its entirety herein by reference.

As shown in FIG. 1, the catheter 10 generally comprises an elongate tubular body 16 extending between a proximal end 12 and a distal functional end 14. The length of the tubular body 16 depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site, as will be understood in the art. In some embodiments, the length of the tubular body 16 is limited by commercial products on the market that can be inserted through the catheter 10. As will be discussed in more detail below, in some embodiments, the length of the tubular body 16 can be at least about 100 cm, at least about 101 cm, at least about 102 cm, at least about 103 cm, at least about 104 cm, at least about 105 cm, at least about 106 cm, at least about 107 cm, at least about 108 cm, at least about 109 cm, at least about 110 cm, between about 100 cm to about 102 cm, between about 102 cm to about 104 cm, between about 104 cm to about 106 cm, between about 106 cm to about 108 cm, between about 108 cm to about 110 cm, less than about 110 cm, and any value in between the ranges listed, including endpoints. This length of the tubular body 16 is shorter and can provide for improved ergonomic hand placement. As well, the length of the tubular body 16 can reduce the excess length controlled by the physician which can help to improve the efficiency of the procedure.

In the illustrated embodiment, the tubular body 16 is divided into at least a fixed proximal section 33 and an axially extendable and retractable distal section 34 separated at a transition 32. FIG. 2 illustrates a side elevational view of the catheter 10 shown in FIG. 1, with the distal segment in a distally extended configuration. In some cases, however, the distal section 34 can include an additional catheter. For example, the distal section 34 can include a catheter, or a portion thereof, configured to extend and be advanced or retracted inside the tubular body 16. In some cases, the distal section 34 can have an outer diameter smaller than an inner diameter of the proximal section 33, thereby allowing the catheter having the distal section 34 to be advanced and/or retracted through the tubular body 16.

The inner diameter of the distal section 34 may be between about 0.030 inches and about 0.112 inches, between about 0.040 inches and about 0.102 inches, between about 0.045 inches and about 0.097 inches, between about 0.050 inches and about 0.092 inches, between about 0.055 inches and about 0.087 inches, between about 0.060 inches and about 0.082 inches, between about 0.062 inches and about 0.080 inches, between about 0.064 inches and about 0.078 inches, between about 0.066 inches and about 0.076 inches, between about 0.068 inches and about 0.074 inches, or between about 0.070 inches and about 0.072 inches.

The inner diameter and the outer diameter of the distal section 34 may be constant or substantially constant along its longitudinal length. Alternatively, the distal section 34 may be tapered near its distal end. The distal section 34 may be tapered at less than or equal to about 1 cm, about 2 cm, about 3 cm, about 4 cm, about 5 cm, about 7 cm, about 10 cm, about 15 cm, about 20 cm, about 23 cm, about 25 cm, about 30 cm, about 31 cm, about 35 cm, about 40 cm, about 45 cm, about 50 cm, about 60 cm, or about 70 cm from its distal end. In some embodiments, the taper may be positioned between about 25 cm and about 35 cm from the distal end of the distal section 34.

The inner diameter of the distal section 34 may be tapered or decreased in the distal direction near the distal end to an internal diameter that is less than or equal to about 95%, about 90%, about 85%, about 80%, about 75%, about 70%, about 65%, about 60%, about 55%, or about 50% of the adjacent, untapered internal diameter. In some embodiments, the internal diameter of the tapered distal section 34 may be between about 50% and about 70% of the adjacent, untapered internal diameter. For example, the untapered internal diameter at the proximal end of the distal section 34 may be about 0.071 inches and the tapered internal diameter at the distal end of the distal section 34 may be about 0.035 inches, 0.045 inches, or 0.055 inches. The inner diameter of the distal section 34 may be tapered or increased near the distal end by greater than or equal to about 102%, 104%, 106%, 108%, or more of the internal diameter just proximal to a transition into the taper. The tapered inner diameter of the distal section 34 may be less than or equal to about 0.11 inches, about 0.1 inches, about 0.090 inches, about 0.080 inches, about 0.070 inches, about 0.065 inches, about 0.060 inches, about 0.055 inches, about 0.050 inches, about 0.045 inches, about 0.040 inches, about 0.035 inches, about 0.030 inches, about 0.025 inches, about 0.020 inches, about 0.015 inches, or about 0.010 inches. In some embodiments, the length of the distal tapered portion of the distal section 34 may be between about 25 cm and about 35 cm, between about 25 cm and about 30 cm, between about 30 cm and 35 cm, or approximately 30 cm.

The length of the distal section 34 may be between about 13 cm and about 53 cm, between about 18 cm and about 48 cm, between about 23 cm and about 43 cm, between about 28 cm and about 38 cm, between about 29 cm and about 39 cm, between about 30 cm and about 40 cm, between about 31 cm and about 41 cm, or between about 32 cm and about 42 cm. The length of the distal section 34 may be less than or equal to about 20 cm, about 25 cm, about 30 cm, about 33 cm, about 35 cm, about 40 cm, about 41 cm, about 45 cm, about 50 cm, about 55 cm, about 60 cm, about 70 cm, or about 80 cm. The length of the distal section 34 may depend on the degree of tapering of the internal diameter of the distal section 34.

In cases where the distal section 34 is part of a separate catheter and/or medical instrument, the catheter including the distal section 34 can have a length such that the distal section 34 can extend between about 13 cm and about 53 cm, between about 18 cm and about 48 cm, between about 23 cm and about 43 cm, between about 28 cm and about 38 cm, between about 29 cm and about 39 cm, between about 30 cm and about 40 cm, between about 31 cm and about 41 cm, or between about 32 cm and about 42 cm, beyond a distal end of the tubular section 16 (e.g., the portion of the tubular body 16 where the transition 32 is located). The distal section 34 of the catheter may be less than or equal to about 20 cm, about 25 cm, about 30 cm, about 33 cm, about 35 cm, about 40 cm, about 41 cm, about 45 cm, about 50 cm, about 55 cm, about 60 cm, about 70 cm, or about 80 cm.

The proximal end 12 of catheter 10 can include a manifold 18 having one or more access ports. In some embodiments, manifold 18 is provided with a proximal port such as a guidewire port 20 in an over-the-wire construction, and at least one side port such as aspiration port 22. Alternatively, the aspiration port 22 may be omitted if the procedure involves removal of the guidewire proximally from the guidewire port 20 following placement of the aspiration catheter, and aspiration through the guidewire port. Additional access ports and lumen may be provided as needed, depending upon the functional capabilities of the catheter. Manifold 18 may be injection molded from any of a variety of medical grade plastics, or formed in accordance with other techniques known in the art.

In some embodiments, the manifold 18 may be provided with a control 24, for controlling the axial position of the distal segment 34 of the catheter. Control 24 may take any of a variety of forms depending upon the mechanical structure and desired axial range of travel of the distal segment 34. In the illustrated embodiment, control 24 includes a slider switch which is mechanically axially movably linked to the distal segment such that proximal retraction of the slider switch 24 produces a proximal movement of the distal segment 34. This retracts the distal segment 34 into the proximal section 33 as illustrated in FIG. 1. Distal axial advancement of the slider switch 24 produces a distal axial advance of the distal segment 34, as illustrated in FIG. 2.

Any of a variety of controls may be utilized, including switches, buttons, levers, rotatable knobs, pull/push wires, and others which will be apparent to those of skill in the art in view of the disclosure herein. The control will generally be linked to the distal segment by a control wire 42.

In some embodiments, the proximal section 33 and distal section 34 maybe provided as separate devices, in which construction the proximal control may be omitted. The distal end of proximal section 33 may be provided with one or more jaws, for morcellating or otherwise breaking thrombus or other obstruction into pieces or otherwise facilitating aspiration. The proximal section 33 may additionally be mechanically coupled to or adapted for coupling to a source of vibrational or rotational movement, such as to provide the intermittent or pulsatile movement discussed elsewhere herein to facilitate navigation into the vasculature. Using axial reciprocation, and/or rotation, and/or biting action of the distal jaws, the clinician may be able to reach the obstruction using proximal section 33. In cases where the distal section 34 is part of a separate catheter, the separate catheter can incorporate any of the features of the distal section 34 disclosed herein to the extent such features are not mutually exclusive with the distal section 34 being part of the separate catheter.

### Overview of Method of Treatment

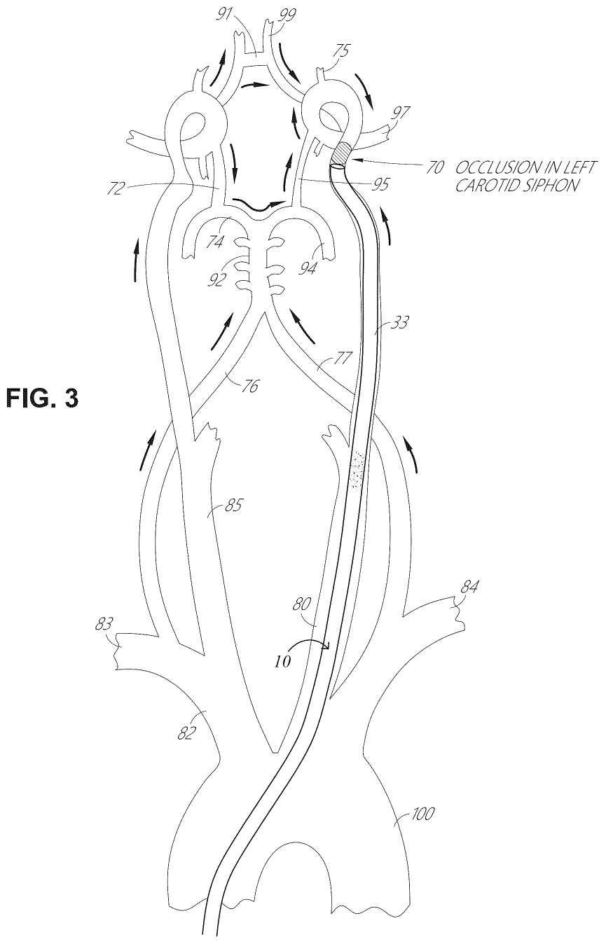

In some embodiments, the proximal section 33 of the catheter 10 is able to reach an obstruction in the left carotid siphon. FIG. 3 illustrates a cerebral arterial vasculature including the Circle of Willis, and an access catheter positioned at an occlusion in the left carotid siphon artery. In some examples, if the proximal section 33 is not able to advance sufficiently close to the obstruction, a separate telescoping distal section 34 may be introduced into the proximal section 33 and advanced therethrough and beyond, as illustrated in FIGS. 2 and 4-6F, to reach the obstruction. In some cases, the proximal section 33 of the catheter 10 may correspond to a guide catheter, and the distal section 34 may correspond to a separate insert catheter, such as insert catheters 1124, 1300, 1300′, 1900, and/or 2260 which are further described herein. In such cases, the insert catheter may be advanced and/or retracted through the guide catheter.

The cerebral circulation is regulated in such a way that a constant total cerebral blood flow (CBF) is generally maintained under varying conditions. For example, a reduction in flow to one part of the brain, such as in acute ischemic stroke, may be compensated by an increase in flow to another part, so that CBF to any one region of the brain remains unchanged. More importantly, when one part of the brain becomes ischemic due to a vascular occlusion, the brain compensates by increasing blood flow to the ischemic area through its collateral circulation.

### Treatment of Acute Occlusion

FIG. 3 depicts cerebral arterial vasculature including the Circle of Willis. Aorta 100 gives rise to right brachiocephalic artery 82, left common carotid artery (CCA) 80, and left subclavian artery 84. The brachiocephalic artery 82 further branches into right common carotid artery 85 and right subclavian artery 83. The left CCA gives rise to left internal carotid artery (ICA) 90 which becomes left middle cerebral artery (MCA) 97 and left anterior cerebral artery (ACA) 99. Anteriorly, the Circle of Willis is formed by the internal carotid arteries, the anterior cerebral arteries, and anterior communicating artery 91 which connects the two ACAs. The right and left ICA also send right posterior communicating artery 72 and left posterior communicating artery 95 to connect, respectively, with right posterior cerebral artery (PCA) 74 and left PCA 94. The two posterior communicating arteries and PCAs, and the origin of the posterior cerebral artery from basilar artery 92 complete the circle posteriorly.

When an occlusion occurs acutely, for example, in left carotid siphon 70, as depicted in FIG. 3, blood flow in the right cerebral arteries, left external carotid artery 78, right vertebral artery 76 and left vertebral artery 77 increases, resulting in directional change of flow through the Circle of Willis to compensate for the sudden decrease of blood flow in the left carotid siphon. Specifically, blood flow reverses in right posterior communicating artery 72, right PCA 74, left posterior communicating artery 95. Anterior communicating artery 91 opens, reversing flow in left ACA 99, and flow increases in the left external carotid artery, reversing flow along left ophthalmic artery 75, all of which contribute to flow in left ICA 90 distal the occlusion to provide perfusion to the ischemic area distal to the occlusion.

As illustrated in FIG. 3, the proximal segment of catheter 10 is transluminally navigated along or over the guidewire, to the proximal side of the occlusion. Transluminal navigation may be accomplished with the distal section 34 of the catheter in the first, proximally retracted configuration. This enables distal advance of the proximal section 33 until further progress is inhibited by small and/or tortuous vasculature. Alternatively, the distal section 34 is a separate device, and is not inserted into the proximal section 33 until it is determined that the proximal section 33 cannot safely reach the occlusion. In the example illustrated in FIG. 3, the occlusion may be safely reached by the proximal section 33, without the need to insert or distally extend a distal section 34.

The distal end of the proximal section 33 of aspiration catheter 10 is inserted typically through an incision on a peripheral artery over a guidewire and advanced as far as deemed safe into a more distal carotid or intracranial artery, such as the cervical carotid, terminal ICA, carotid siphon, MCA, or ACA. The occlusion site can be localized with cerebral angiogram or IVUS. In emergency situations, the catheter can be inserted directly into the symptomatic carotid artery after localization of the occlusion with the assistance of IVUS or standard carotid doppler and TCD.

If it does not appear that sufficient distal navigation of the proximal section 33 to reach the occlusion can be safely accomplished, the distal section 34 is inserted into the proximal port 20 and/or distally extended beyond proximal section 33 until the distal tip 38 is positioned in the vicinity of the proximal edge of the obstruction.

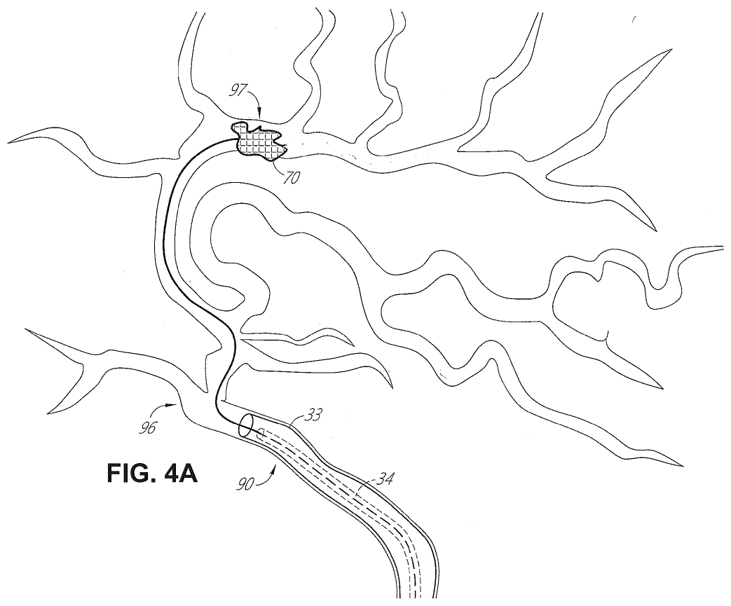

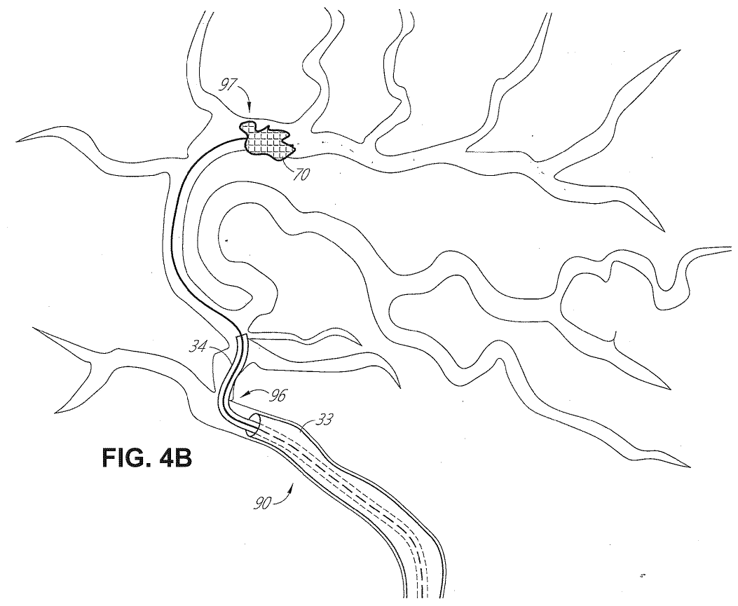

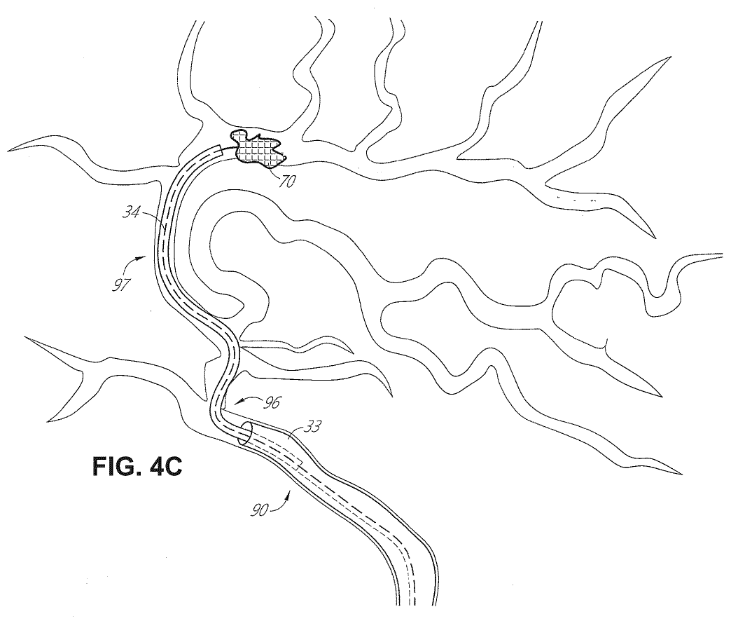

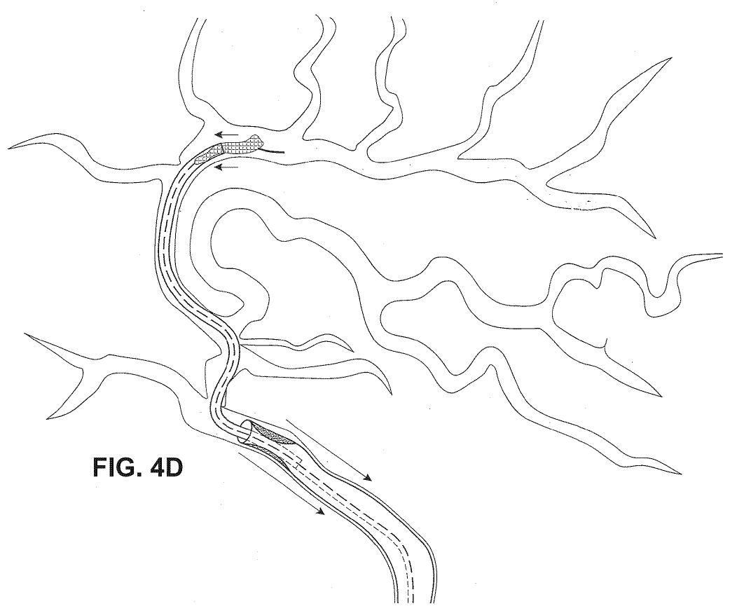

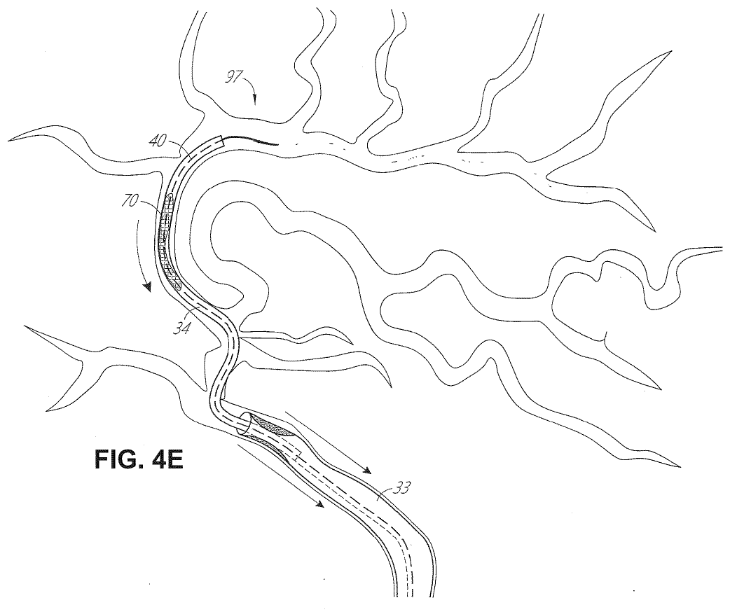

Referring to FIG. 4A, an obstruction 70 is lodged in the middle cerebral artery 97. Proximal section 33 is positioned in the ICA and not able to navigate beyond a certain point such as at the branch 96 to the MCA artery 97. The proximal section 33 may be provided with a distal section 34 carried therein. Alternatively, a separate distal section 34 may be introduced into the proximal end of proximal section 33 once the determination has been made that the obstruction 70 cannot be reached directly by proximal section 33 alone. As seen in FIGS. 4B and 4C, the distal section 34 may thereafter be transluminally navigated through the distal tortuous vasculature between proximal section 33 and the obstruction 70.

As shown in FIG. 4D, the obstruction 70 may thereafter be drawn into distal section 34. In some embodiments, the obstruction 70 can be drawn into the distal section 34 upon application of constant or pulsatile negative pressure. In some examples, the distal end of the distal section 34 can include jaws or other mechanical features for with or without the use of jaws or other activation on the distal end of distal section 34. FIG. 4E illustrates that once the obstruction 70 has either been drawn into distal section 34, or drawn sufficiently into distal section 34, the proximal section 33 and distal section 34 are thereafter proximally withdrawn. In cases where an insert catheter and/or a guide catheter are used, the guidewire and/or the insert catheter can be retracted before applying aspiration via the guide catheter.

### Treatment of Occlusion in Internal Carotid Artery

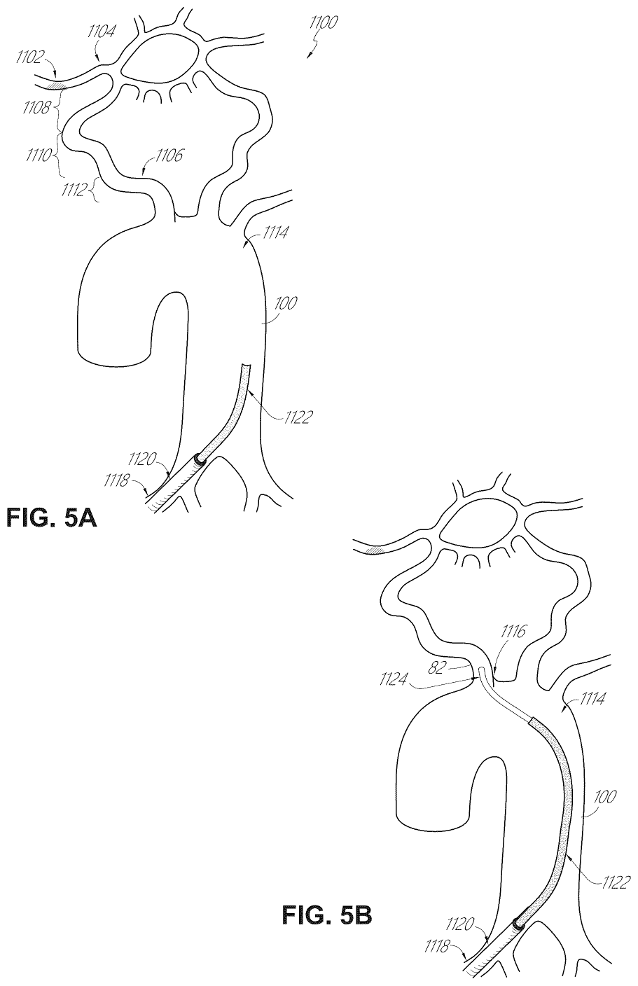

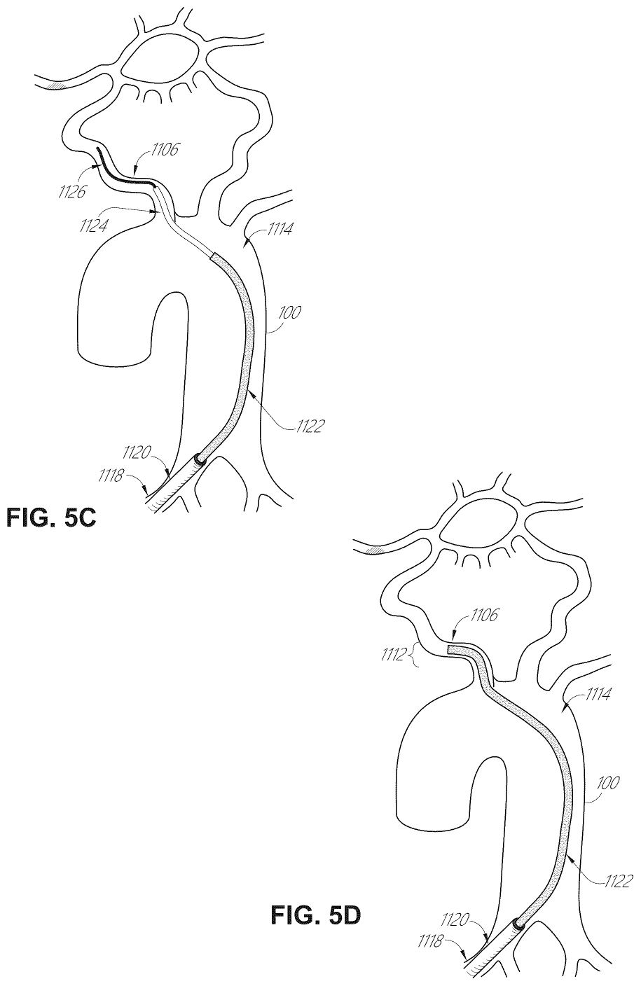

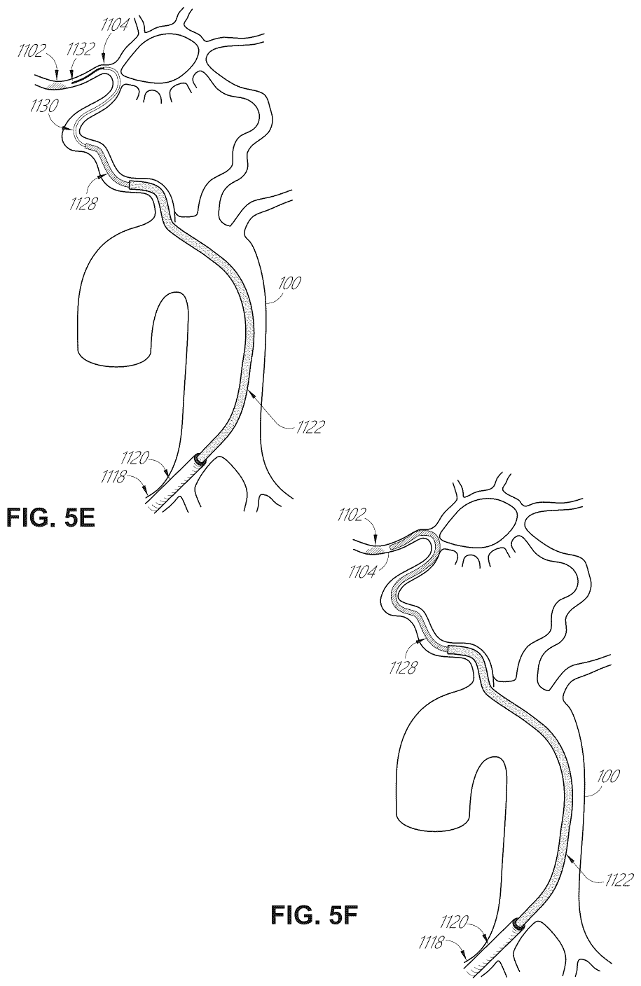

FIGS. 5A-5F illustrates accessing an occlusion positioned in the right middle cerebral artery of the right internal carotid artery. As shown, the cerebral circulation 1100 is simplified for the case of demonstrating procedural steps. A thrombotic occlusion or clot 1102 is in the right middle cerebral artery (RMCA) 1104. The RMCA 1104 branches from the right internal carotid artery (RICA) 1106. The RICA 1106 branches from the right common carotid artery (RCCA) (not shown). The RICA 1106 comprises cerebral 1108 (most distal from the aorta 100), cavernous 1110, and petrous 1112 (most proximal from the aorta 100) segments. The RCCA branches from the brachiocephalic artery. The brachiocephalic artery branches from the aortic arch 1114 of the aorta 100.

The procedural steps for aspirating a thrombotic occlusion are described as follows. Referring to FIG. 5A, an introducer sheath 1120 is introduced at the femoral artery 1118. The outer diameter of the introducer sheath 1120 may be equal to or less than about 12 F, 11 F, 10 F, 9 F, 8 F, 7 F, or 6 F. Then, a guide sheath 1122 is inserted through the introducer sheath 1120. The outer diameter of the guide sheath 1122 may be equal to or less than about 9 F, 8 F, 7 F, 6 F, 5 F, 4 F, or 3 F, and the inner diameter of the introducer sheath 1120 may be greater than the outer diameter of the guide sheath 1122.

Referring to FIG. 5B, an insert catheter 1124 is inserted through the guide sheath 1122. The outer diameter of the insert catheter 1124 may be equal to or less than about 9 F, 8 F, 7 F, 6 F, 5 F, 4 F, or 3 F, and the inner diameter of the guide sheath 1122 may be greater than the outer diameter of the insert catheter 1124. In some cases, a first guidewire 1126 may be introduced through the insert catheter 1124 (not shown in FIG. 5B). Then, the guide sheath 1122, the insert catheter 1124, and optionally the first guidewire 1126 are tracked up to the aortic arch 1114. The insert catheter 1124 is used to engage the origin of a vessel. In FIG. 5B, the insert catheter 1124 engages the origin 1116 of the brachiocephalic artery 82. An angiographic run is performed by injecting contrast media through the insert catheter 1124. In the cases where the first guidewire 1126 is used before the angiographic run, the first guidewire 1126 is removed prior to injecting the contrast media.

Referring to FIG. 5C, the first guidewire 1126 is inserted through the lumen of the insert catheter 1124. Then, the first guidewire 1126, the insert catheter 1124, and the guide sheath 1122 are advanced together to the RICA 1106. Referring to FIG. 5D, due to the stiffness of a typical guide sheath 1122 currently available in the market (e.g., Neuron MAX System produced by Penumbra Inc.), the most distal vessel that the guide sheath 1122 could navigate to is the petrous segment 1112 of the RICA 1106. Once the first guidewire 1126, the insert catheter 1124, and the guide sheath 1122 are advanced to the RICA 1106, both the first guidewire 1126 and the insert catheter 1124 are removed.

Referring to FIG. 5E, a second guidewire 1132 loaded inside the central lumen of a reperfusion catheter 1130 (e.g., 3Max), which is loaded inside the central lumen of an aspiration catheter 1128 (e.g., ACE 68), are introduced through the guide sheath 1122. The diameter of the second guidewire 1132 may be equal to or less than about 0.03″, about 0.025″, about 0.02″, about 0.016″, about 0.014″, about 0.01″, or about 0.005″. The inner diameter of the reperfusion catheter 1130 may be greater than the outer diameter of the second guidewire 1132. The inner diameter of the aspiration catheter 1128 may be greater than the outer diameter of the reperfusion catheter 1130. The inner diameter of the guide sheath 1122 may be greater than the outer diameter of the aspiration catheter 1128. Then, the second guidewire 1132 is advanced distally and positioned at the proximal end of the clot 1102 in the RMCA 1104.

Referring to FIG. 5F, the aspiration catheter 1128 is tracked over the reperfusion catheter 1130 and the second guidewire 1132 to the proximal end of the clot 1102 in the RMCA 1104. Both the second guidewire 1132 and the reperfusion catheter 1130 are removed. A vacuum pressure is then applied at the proximal end of the aspiration catheter 1128 to aspirate the clot 1102 through the central lumen of the aspiration catheter 1128.

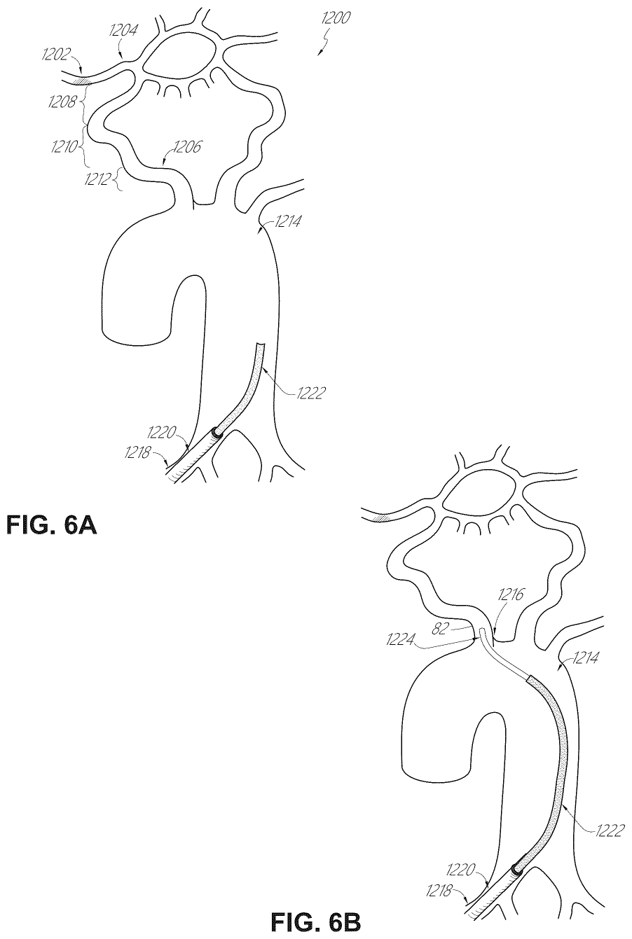

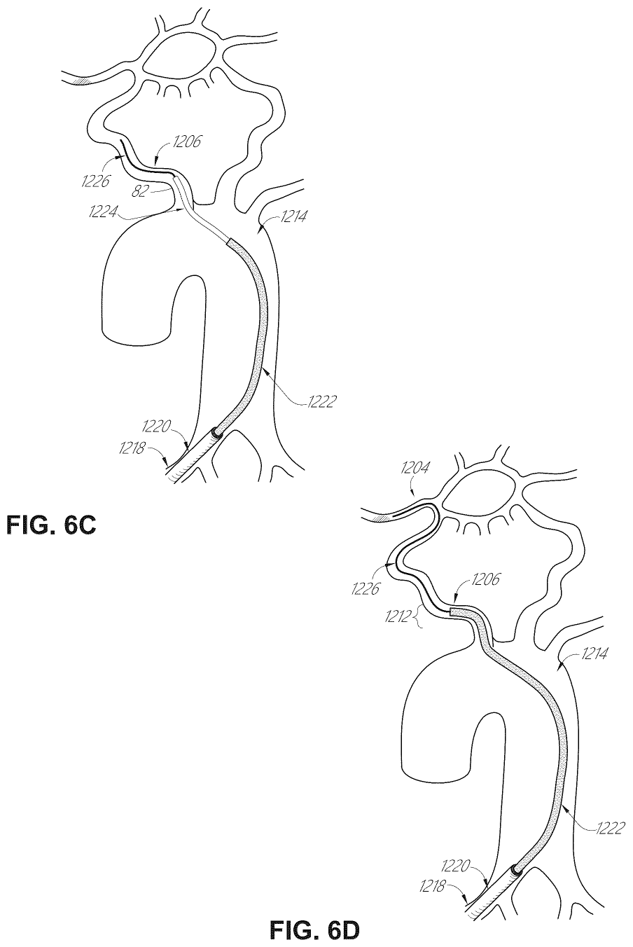

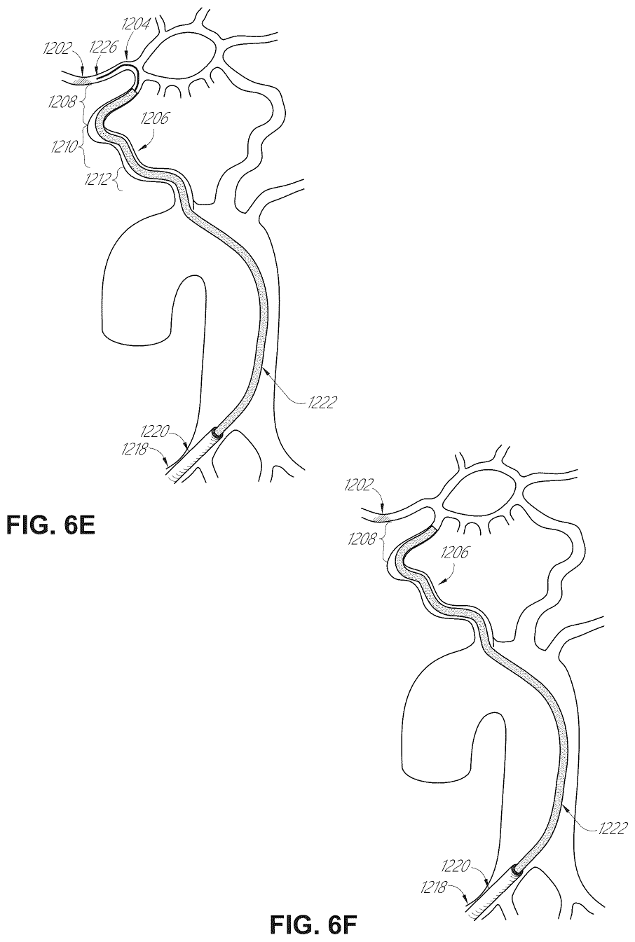

FIGS. 6A-6F illustrate an alternative and simplified method for aspirating a thrombotic occlusion. The alternative steps for aspirating a thrombotic occlusion make use of a transitional guidewire and a transitional guide sheath. The transitional guidewire has a soft and trackable distal segment with a smaller diameter so that the transitional guidewire may be advanced deeper than the guidewire 1126 described in FIG. 5C. In addition, the transitional guide sheath has a soft and trackable distal segment such that the transitional guide sheath may be advanced deeper than the guide sheath 1122 described in FIG. 5D. Using a transitional guidewire and a transitional guide sheath that can be advanced to an area near the clot eliminates the need to use a second guidewire or a reperfusion catheter to reach the clot.

Referring to FIG. 6A, an introducer sheath 1220 is introduced at the femoral artery 1218. The outer diameter of the introducer sheath 1220 may be equal to or less than about 12 F, 11 F, 10 F, 9 F, 8 F, 7 F, or 6 F. Then, a transitional guide sheath 1222 such as the combination access and aspiration catheter discussed in greater detail below is inserted through the introducer sheath 1120 at the femoral artery 1218. The outer diameter of the guide sheath 1222 may be equal to or less than about 9 F, 8 F, 7 F, 6 F, 5 F, 4 F, or 3 F. Referring to FIG. 6B, an insert catheter 1224 is inserted through the transitional guide sheath 1222. The outer diameter of the insert catheter 1224 may be less than about 9 F, 8 F, 7 F, 6 F, 5 F, 4 F, or 3 F, and the inner diameter of the transitional guide sheath 1222 may be greater than the outer diameter of the insert catheter 1224. In some cases, a first guidewire may be introduced through the insert catheter 1224 (not shown in FIG. 6B). The diameter of the proximal section of the first guidewire may be equal to or less than about 0.079″, about 0.066″, about 0.053″, about 0.038″, about 0.035″, about 0.030″, or about 0.013″.

The transitional guide sheath 1222, the insert catheter 1224, and optionally the first guidewire are tracked up to the aortic arch 1214. See FIG. 6B. The insert catheter 1224 may be used to select the origin of a vessel. In FIG. 6B, the insert catheter 1224 engages the origin 1216 of the brachiocephalic artery 82. An angiographic run may be performed by injecting contrast media through the insert catheter 1224. In the cases where the first guidewire is used before the angiographic run, the first guidewire is preferably removed prior to injecting the contrast media.

Referring to FIG. 6C, the transitional guidewire 1226 is inserted through the lumen of the insert catheter 1224 or guide sheath 1222. The diameter of at least a portion of the transitional guidewire 1226 (e.g., proximal diameter) is substantially similar to that of the first guidewire 1126. The diameter of at least a portion of the transitional guidewire 1226 (e.g., distal diameter) may be smaller than that of the first guidewire 1126 and may have a diameter along a proximal segment of at least about 0.030″ and in one implementation about 0.038″. A transition begins within the range of from about 15 cm-30 cm from the distal end, and typically no more than about 20 cm or 25 cm from the distal end, distally of which the diameter tapers down to no more than about 0.018″ and in one implementation about 0.016″. Referring to FIG. 6D, if utilized, the insert catheter 1224 may be removed because it is too stiff to be advanced to the MCA 1204. In some embodiments, the transitional guidewire 1226 provides sufficient back up support that the combination access and aspiration catheter 1224 may be advanced directly over the transitional guidewire without any intervening devices. Then, the transitional guidewire 1226 is advanced to the MCA 1204. The transitional guidewire 1226 has a distal segment that has a smaller diameter than that of the first guidewire 1126 described in FIG. 5C. The distal segment of the transitional guidewire 1226 comprises a soft and atraumatic tip and can be tracked to the remote neurovasculature such as the MCA 1204, which is distal to the petrous segment 1212 of the ICA 1206.

Referring to FIG. 6E, the transitional guide sheath 1222 is advanced to or beyond the cavernous segment 1210 or the cerebral segment 1208 of the ICA 1206. Unlike the guide sheath 1122 described in FIG. 5D, the transitional guide sheath 1222 may be advanced to the cavernous segment 1210 or the cerebral segment 1208 of the ICA 1206 beyond the petrous segment 1212 because the transitional guide sheath 1222 has a soft yet trackable distal segment. The larger proximal diameter and stiffer body of the transitional guidewire 1226 may provide better support for the transitional guide sheath 1222 to track through the vasculature.

Referring to FIG. 6F, after the transitional guide sheath 1222 is advanced to the cerebral segment 1208 of the ICA 1206, the transitional guidewire 1226 is removed. Then, a vacuum pressure is applied at the proximal end of the transitional guide sheath 1222 to aspirate the clot 1202 through the central lumen of the transitional guide sheath 1222. The inner diameter of the transitional guide sheath 1222 may be equal to about or greater than about 0.100″, about 0.088″, about 0.080″, about 0.070″, or about 0.060″. The inner diameter of the transitional guide sheath 1222 is larger than the aspiration catheter 1128 described in FIG. 5E, which translates to more effective aspiration. The cross-sectional area of the central lumen of the transitional guide sheath 1222 may be almost twice as large as that of the largest aspiration catheter 1128 currently available.

If the guide sheath 1222 is not able to track deep enough into the distal vasculature to reach the clot or other desired target site, a telescopic extension segment as discussed elsewhere herein may be introduced into the proximal end of sheath 1222 and advanced distally to extend beyond the distal end of the sheath 1222 and thereby extend the reach of the aspiration system. In some embodiments, the extension segment has an ID of about 0.070″.

If thrombotic material is not able to be drawn into the sheath 1222 or extension segment under constant vacuum, pulsatile vacuum may be applied. If pulsatile vacuum does not satisfactorily capture the clot, an agitator may be advanced through the sheath 1222 and extension segment to facilitate drawing the clot into the central lumen.

### Insert Catheters

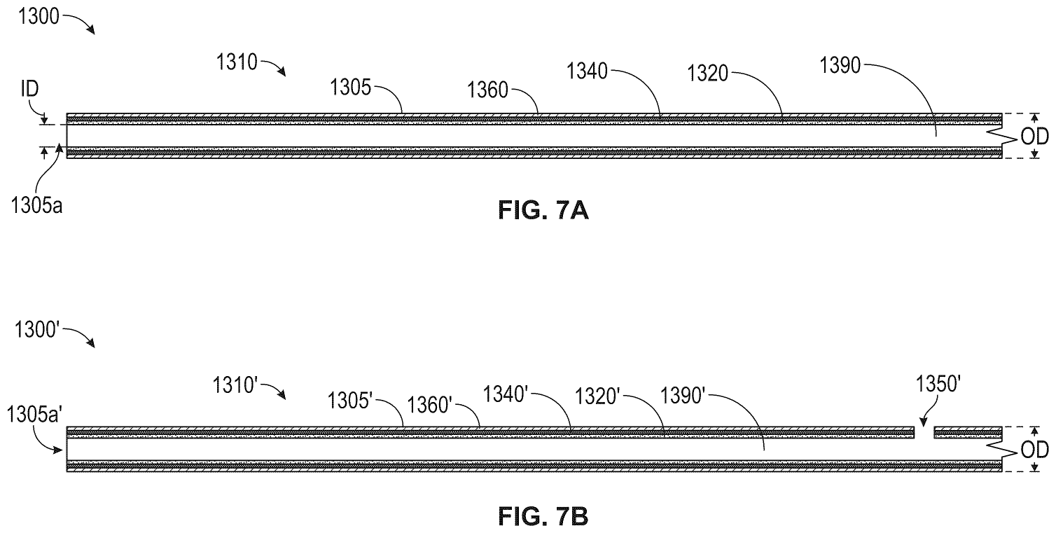

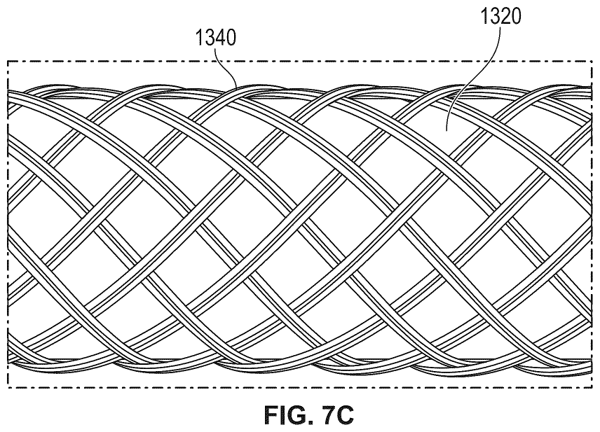

Any of the catheters disclosed herein may be provided with an inner liner, a reinforcing element, and/or an outer jacket. Referring to FIG. 7A, a distal portion 1310 of an insert catheter 1300 having a tubular body 1305 including a distal end 1305a, an inner liner 1320, a reinforcing element 1340, and an outer jacket 1360. In some cases, the reinforcing clement 1340 can include a braid and/or a spring coil, such as a braid as shown in FIG. 7C. The reinforcing element 1340 can be positioned radially outward of the inner liner 1320. In some cases, the reinforcing element 1340 can also be embedded in the outer jacket 1360, which can be positioned radially outward of the inner liner 1320.

The insert catheter 1300 can also include a lumen 1390 extending radially inward from the inner liner 1320. The lumen 1390 can extend an entire length of the insert catheter 1300. The lumen 1390 can beneficially receive other interventional devices, such as a guidewire. In some cases, the distal portion 1310 may be radiopaque, which can beneficially allow visualization of the insert catheter 1300 under fluoroscopy.

The reinforcing element 1340 and/or the outer jacket 1360 may extend an entire length of the inner liner 1320 along the distal portion 1310. In some cases, however, the reinforcing element 1340 and/or the outer jacket 1360 may extend less than entire length of the inner liner 1320 along the distal portion 1310. For example, the reinforcing element 1340 can extend distally along the distal portion 1310 and end about 10 mm, 15 mm, 20 mm, 40 mm, 50 mm, 60 mm, 70 mm, 75 mm, 80 mm, 85 mm, 90 mm, 100 mm, 120 mm, and/or 150 mm from the distal end 1305a. When the reinforcement element 1340 does not extend an entire length of the inner liner 1320, the distal-most portion of the insert catheter 1300 does not include a reinforcing element 1340.

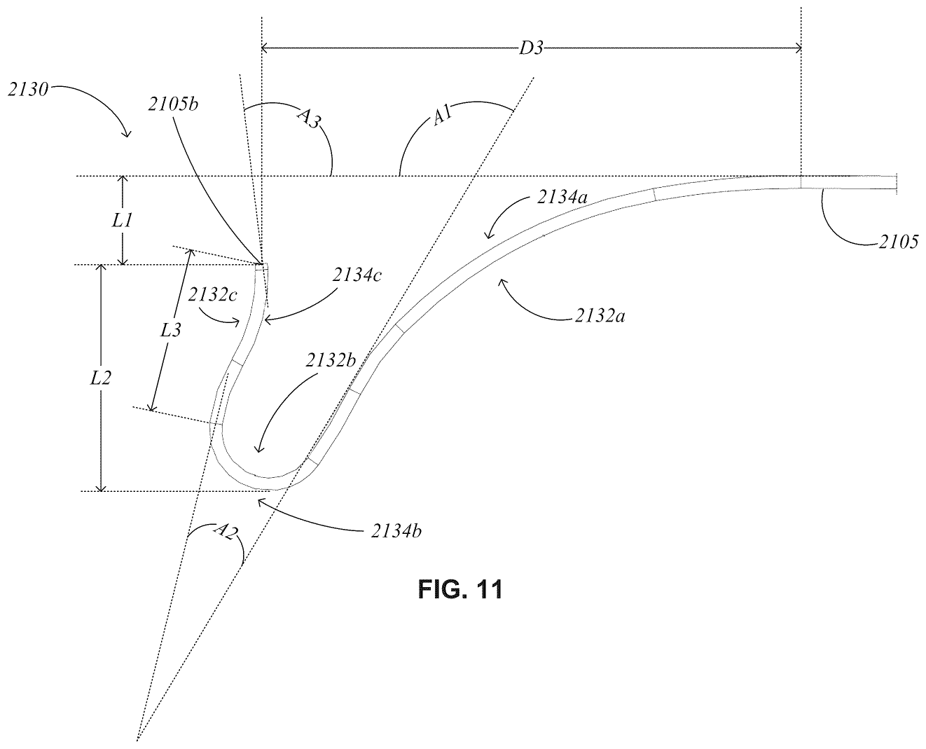

The outer jacket 1360 may include a plurality of tubular segments positioned coaxially over the inner liner 1320 and/or the reinforcing element 1340. In some cases, the outer jacket 1360 can include at least two tubular segments, and in some cases 3, 4, 5, 6, 7, 8, 9, and/or 10 tubular segment. The plurality of tubular segments of the outer jacket 1360 can include durometers that decrease in a distal direction. The durometers of the plurality of tubular segments can range from about 35 D to about 75 D. The plurality of tubular segments of the outer jacket 1360 can have stiffness that decreases (that is, increasingly more flexible) in a distal direction, such as described further below with reference to FIG. 11.

In some cases, the insert catheter 1300 can include an outer diameter OD ranging from about 0.020 in. to about 0.12 in. For example, the outer diameter OD of the insert catheter 1300 can be from about 0.030 in. to about 0.11 in., from about 0.040 in. to about 0.10 in., from about 0.050 in. to about 0.09 in., from about 0.060 in. to about 0.090 in., or from about 0.070 in. to about 0.080 in. The outer diameter OD of the insert catheter 1300 may be constant through an entire length of the insert catheter 1300. In some cases, however, the outer diameter OD of the insert catheter 1300 may vary along different portions of the insert catheter 1300. A distal-most portion of the insert catheter 1300 may taper in a distal direction (that is, increasingly smaller towards the distal end 1305a). That is, the outer diameter OD of the insert catheter 1300 on the distal end 1305a may be smaller than the outer diameter OD along portions of the insert catheter 1300 proximal to the distal end 1305a.

The insert catheter 1300 can include an inner diameter ID (corresponding to the diameter of the lumen 1390) ranging from about 0.010 in. to about 0.070 in. For example, the inner diameter ID of the insert catheter 1300 can be from about 0.015 in. to about 0.065 in., from about 0.020 in. to about 0.060 in., from about 0.025 in. to about 0.055 in., from about 0.030 in. to about 0.050 in., and/or from about 0.035 in. to about 0.045 in. The inner diameter ID of the insert catheter 1300 may be constant through an entire length of the insert catheter 1300.

The insert catheter 1300 can have a length ranging from about 100 cm to about 170 cm. In some cases, the length of the insert catheter 1300 can be from about 110 cm. to about 160 cm., from about 120 cm. to about 150 cm., from about 130 cm, to about 145 cm, and/or from about 135 cm. to about 145 cm. The insert catheter 1300 can be longer than a guide catheter and/or sheath which the insert catheter 1300 can extend through.

The insert catheter 1300 may include a pre-shaped tip. For example, the pre-shaped tip of the insert catheter 1300 may include one or more concave and/or convex portions, and/or one or more curves in various directions. In some cases, at least portions of the pre-shaped tip can establish an angle relative to a longitudinal axis of the insert catheter 1300. The pre-shaped tip can beneficially allow for easier and faster access, insertion, and/or steerability of the insert catheter along tortuous and/or elongated vessels such as the aortic arch vessels. For example, the insert catheter 1300 can be advanced at least as far as the aortic arch, ostia of the aortic arch, and/or further into the vasculature (e.g., the right carotid artery).

In some cases, a stiffness rating of the insert catheter 1300 may vary along the length of the insert catheter 1300. For example, distal portions of the insert catheter 1300 may be softer than more proximal portions of the insert catheter 1300. Having a softer distal portion 1310 can beneficially prevent the insert catheter from damaging vessel walls when, for example, the insert catheter 1300 is advanced through the vasculature. The stiffer proximal portions, however, provide support which can beneficially facilitate pushability and/or allow the insert catheter 1300 to stay in place when not actively being advanced or retracted.

It may be desirable for the insert catheter to have a minimum threshold torque response. The minimum threshold torque response can allow rotational movement of the distal portion 1310 of the insert catheter 1300 to more closely replicate rotation applied to a proximal portion of the insert catheter 1300. This can beneficially allow for more predictable steerability and positioning of the insert catheter 1300 within the vasculature of a patient. For example, in some cases, the insert catheter 1300 can have an average torque response from about 0.001 Ncm/deg to about 0.005 Ncm/deg. In some cases, the insert catheter 1300 can have an average torque response from about 0.0012 Ncm/deg to about 0.0048 Ncm/deg, from about 0.002 Ncm/deg to about 0.0044 Ncm/deg, and/or from about 0.003 Ncm/deg to about 0.004 Ncm/deg. In some cases, the insert catheter 1300 can have a torque response from about 0.01 Ncm/deg to about 1.5 Ncm/deg. For example, the insert catheter 1300 can have a torque response from about 0.05 Ncm/deg to about 1.2 Ncm/deg, from about 0.1 Ncm/deg to about 0.9 Ncm/deg, and/or from about 0.3 Ncm/deg to about 0.7 Ncm/deg.

In some cases, the insert catheter 1300 can be used to deliver contrast media, saline, drugs, and/or other types of fluids into the vasculature of a patient. The insert catheter 1300 can be used to deliver contrast media, saline, drugs, and/or other types of fluids into the vasculature of a patient with or without a guidewire inserted in the lumen of the insert catheter 1300. In some cases, the insert catheter 1300 can inject fluids (with or without the guidewire inside the lumen of the insert catheter 1300) at a pressure of up to 1500 PSI. For example, the insert catheter 1300 can inject fluids at a pressure of about 1400 PSI, 1300 PSI, 1200 PSI, 1100 PSI, 900 PSI, 800 PSI. In some cases, the insert catheter 1300 can inject fluids at a flow rate of at least about 5 mL/s. For the insert catheter 1300 can inject fluids at a flow rate of about 5 mL/s, 8 mL/s, 10 mL/s, 15 mL/s, 20 mL/s, 30 mL/s, 50 mL/s, 100 mL/s, etc. The insert catheter 1300 can incorporate any features of the other insert catheters disclosed herein.

Any of the insert catheters disclosed herein can be rapid exchange catheters. Rapid exchange catheters can include an exit port positioned distal to a proximal end of the catheter. For example, an insert catheter 1300′ can include an exit port 1350′, as shown in FIG. 7B. The insert catheter 1300′ can incorporate any of the features of the insert catheter 1300 except the differences described with reference to FIG. 7B. For example, the insert catheter 1300′ can include a tubular body 1305′ including a distal end 1305a′, an inner liner 1320′, a reinforcing element 1340′, an outer jacket 1360′, a distal portion 1310′, and/or a lumen 1390′.

In some cases, the exit port 1350′ can be located on an inner (for example, concave) side of a pre-shaped tip of the insert catheter 1300′. The exit port 1350′ can be located on a same side as a concave side of a pre-shaped tip. In some cases, the exit port 1350′ can be in fluid communication with the lumen 1390′. In other implementations, the exit port 1350′ can be located elsewhere around a circumference of the catheter (e.g., on the convex side of a pre-shaped tip). Other example insert catheters, including other insert catheters with different pre-shaped tips, can incorporate any features of the insert catheter 1300′. In some cases, the exit port 1350′ can be located about 10 cm to about 100 cm, about 20 cm to about 90 cm, about 30 cm to about 80 cm, about 40 cm to about 70 cm, or about 50 cm to about 60 cm, from the distal end 1305a′ of the insert catheter 1300′. In some implementations, any of the insert catheter examples disclosed herein, for example, the insert catheter 1900, can incorporate the features of the insert catheter 1300′.

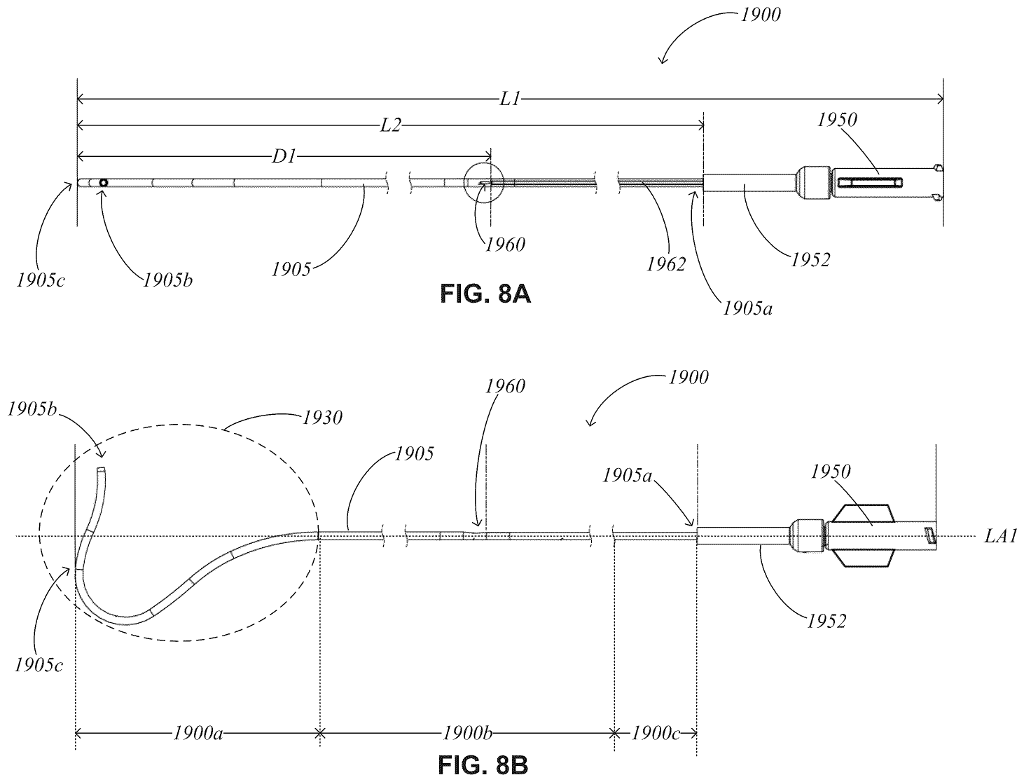

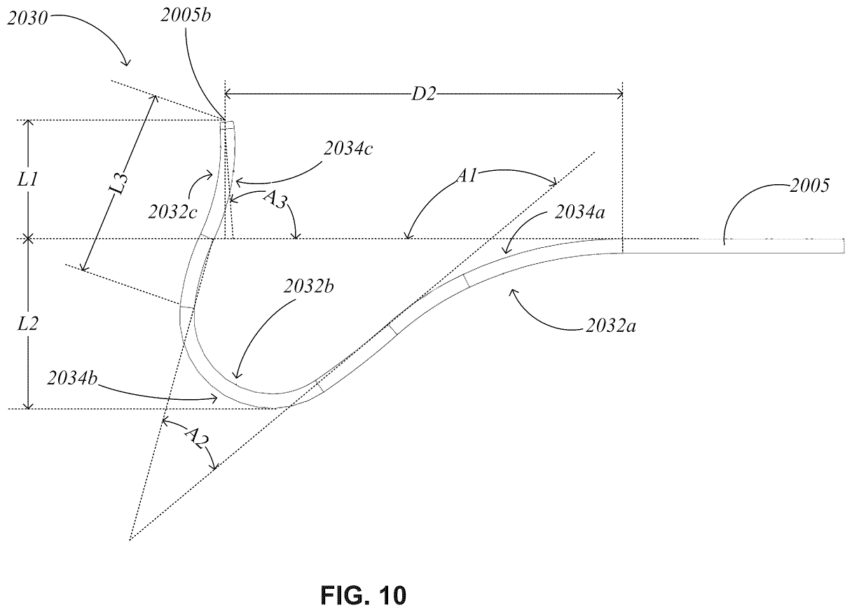

FIGS. 8A and 8B show an example of an insert catheter 1900. The insert catheter 1900 can incorporate any features of the other insert catheter examples disclosed herein. The insert catheter 1900 can include a distal portion 1900a, a transitional portion 1900b, and a proximal portion 1900c. The insert catheter 1900 can include a tubular body 1905 having a proximal end 1905a and a distal end 1905b. The tubular body 1905 can include an inner liner, a reinforcing element, an outer jacket, and/or a lumen 1910 extending between the proximal end 1905a and the distal end 1905b. The inner liner, reinforcing element, and/or outer jacket of the insert catheter 1900 can be similar or identical to the inner liner 1320, reinforcing element 1340, and/or outer jacket 1360 of the insert catheter 1300, which is described in relation to FIGS. 7A-7C. The insert catheter 1900 can include a pre-shaped tip 1930. Additional details regarding the pre-shaped tips are described in relation to FIGS. 10 and 11.

In some implementations, the proximal end 1905a of the tubular body 1905 can be connected to another medical device. For example, the proximal end 1905a of the tubular body 1905 can be connected to a manifold or hub 1950. In some implementations, a strain relief 1952 can be positioned between the proximal end 1905a of the tubular body 1905 and the hub 1950. The strain relief 1952 can beneficially prevent kinking of the tubular body 1905.

The insert catheter 1900 can have a total length L1 (as shown in the top view of FIG. 8A) from a distal-most end of the pre-shaped tip 1930 (see also FIG. 8B) to a proximal end of the hub 1950. L1 can be from about 140 cm to about 200 cm. For example, the insert catheter 1900 can have a total length L1 from about 150 cm to about 190 cm, from about 160 cm to about 180 cm, and/or from about 165 cm to about 175 cm. In some cases, the tubular body 1905 can have a length L2 (as shown in the top view of FIG. 8A) from a distal-most end of the pre-shaped tip 1930 (see also FIG. 8B) to the proximal end 1905a of the tubular body 1905. L2 can be from about 135 cm to about 195 cm. For example, the tubular body 1905 can have a length L2 from about 145 cm to about 185 cm, from about 155 cm to about 175 cm, and/or from about 160 cm to about 170 cm.

The insert catheter 1900 can include a side port 1960. The side port 1960 can be located along the tubular body 1905 of the insert catheter 1900. The side port 1960 can be on a radial/circumferential location of the tubular body 1905 so as to face the same direction as the opening of the lumen of the tubular body 1905 at the distal end 1905b. In the illustrated embodiment, the side port 1960 can be located on a same side as a concave side of the second curve of the pre-shaped tip 1930 (which will be described in greater details below) of the insert catheter 1900. However, the side port 1960 can be located elsewhere around a circumference of the catheter (e.g., on the same side as a convex side of a certain curve of a pre-shaped tip, facing an opposite side as the opening of the distal end 1905b of the tubular body 1905, etc.). The side port 1960 and the lumen 1910 of the insert catheter 1900 can be in fluid communication with each other. This can allow devices to be advanced and/or retracted through the side port 1960, which is closer to the distal end of the insert catheter 1900 than an access location at the hub 1950. For example, a guidewire can be advanced through and/or retracted from the lumen 1910 of the tubular body 1905 via the side port 1960.

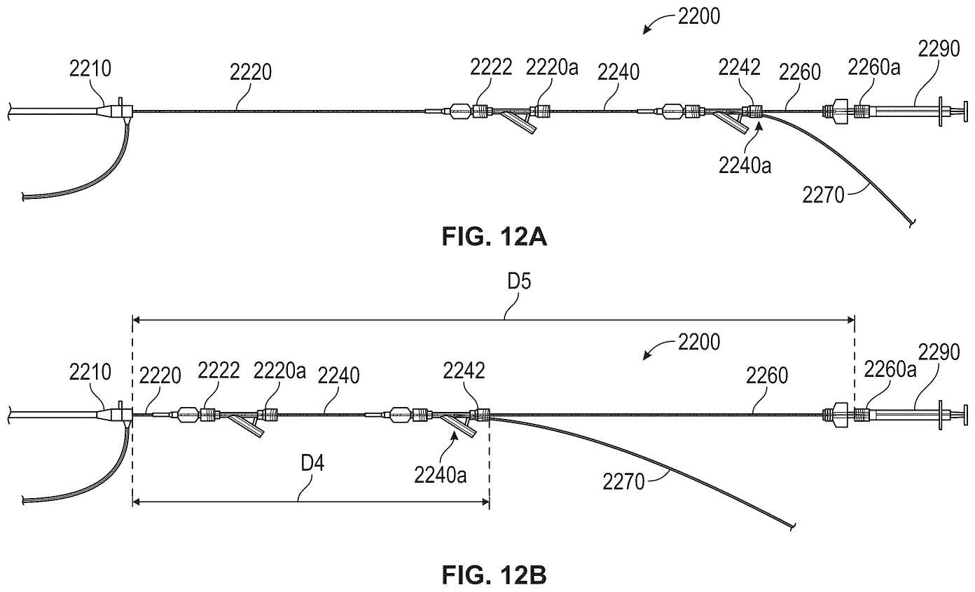

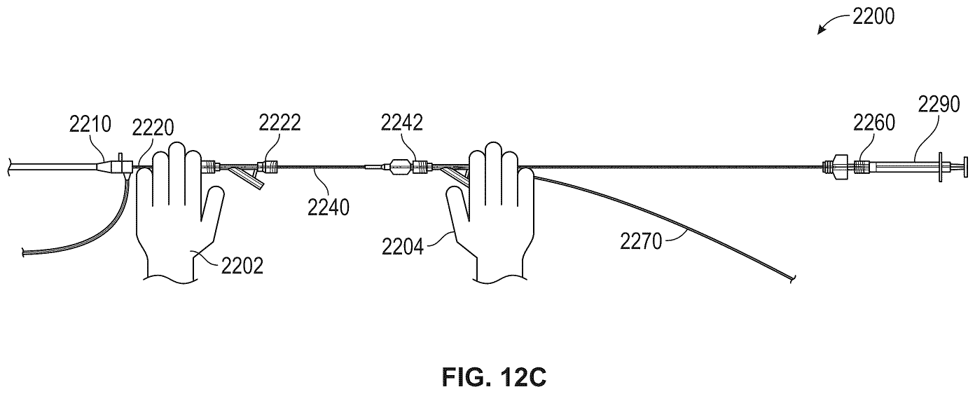

In some cases, the side port 1960 can be positioned proximal to the distal end 1905b of the tubular body 1905. For example, the distal-most location 1905c of the pre-shaped tip 1930 and the side port 1960 can be separated by a distance D1. In the illustrated embodiment, the distal-most location 1905c of the pre-shaped tip 1930 is distal of the distal end 1905b of the tubular body 1905 and can define the distal end of the insert catheter 1900. In some cases, the distance D1 can be from about 25 cm to about 190 cm. The distance D1 can be from about 35 cm to about 180 cm, from about 70 cm to about 150 cm, from about 75 cm to about 125 cm, from about 80 cm to about 120 cm, from about 90 cm to about 110 cm, and/or from about 95 cm to about 105 cm. As further described in relation to FIGS. 12A-12C, the position of the side port 1960 relative to the distal end 1905b of the tubular body 1905 can allow for easier and more efficient control of all interventional devices when controlling more than one interventional device.

As shown in FIG. 8A, the insert catheter 1900 can include a rail 1962. The rail 1962 and the lumen 1910 can extend parallel to each other. The rail 1962 can extend from the side port 1960 to the proximal end 1905a of the tubular body 1905. The rail 1962 can include a groove extending along a length of the tubular body 1905. In some cases, the rail 1962 can have a shape and/or size substantially matching that of at least a portion of a guidewire. The matching shape and/or size can accommodate the portion of the remaining guidewire proximal to the side port 1960 when the guidewire is placed into the lumen 1910 of the insert catheter 1900 via the side port 1960, so that the remaining guidewire can stay at least partially within the rail 1962 rather than staying loose relative to the tubular body 1905. When portions of the tubular body 1905 proximal to the side port 1960 are positioned inside another interventional device, such as a guide sheath 1222 as shown in FIGS. 6A-6F, the rail 1962 can route, organize, support, and/or align the remaining guidewire extending proximal to the side port 1960 along the length of the tubular body 1905 in the annular space between an interior of the interventional device and the insert catheter 1900. In other words, the rail 1962 can perform a function analogous to cord management, such as a cord channel.

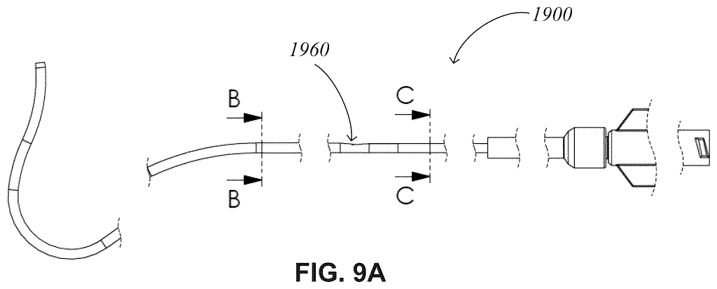

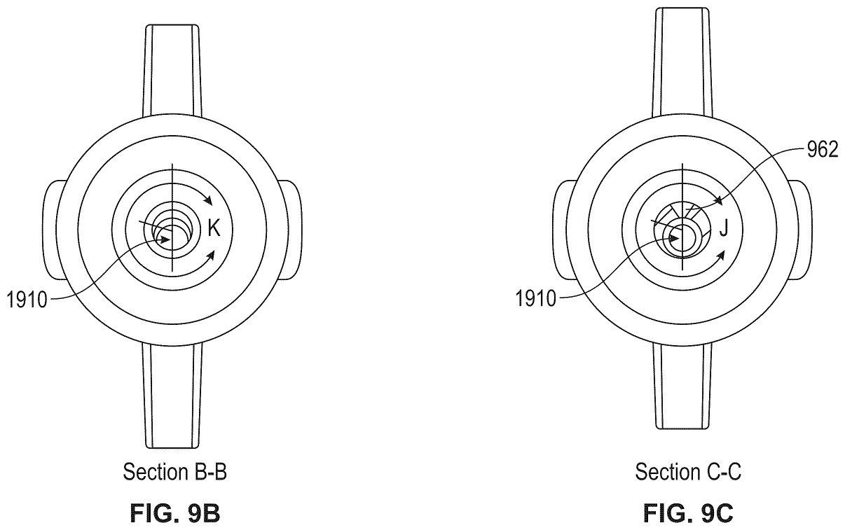

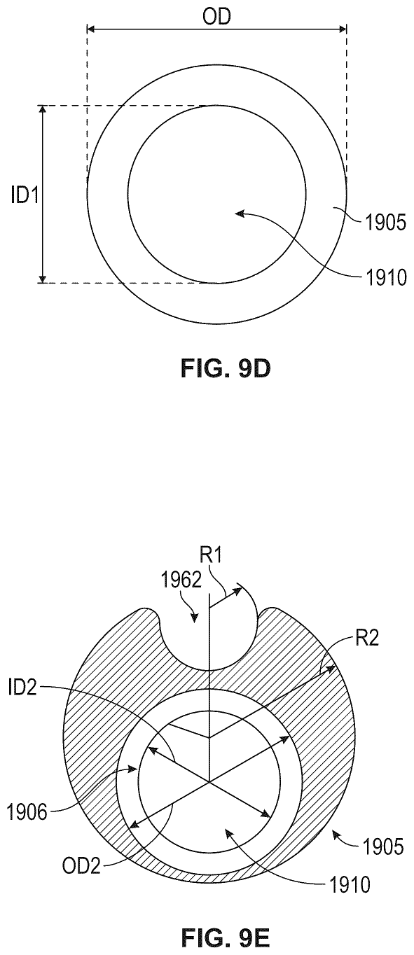

FIGS. 9B and 9C illustrate two cross sections of the insert catheter 1900. FIG. 9B shows a cross section along the line B-B shown in FIG. 9A, while FIG. 9C shows a cross section along the line C-C shown in FIG. 9A. The line B-B can be distal to the side port 1960 and the line C-C can be proximal to the side port 1960. The lumen 1910 of the tubular body 1905 can extend an entire length of the insert catheter 1900. As shown in FIG. 9C, the rail 1962 and the lumen 1910 can extend parallel to each other.

As shown in FIG. 9D, the lumen 1910 of the tubular body 1905 at a location close to or immediately proximal of the pre-shaped tip 1930 (e.g., distal to the side port 1960) can have a diameter ID1 from about 0.015 in. to about 0.1 in. For example, the lumen 1910 can have a diameter ID1 from about 0.020 in. to about 0.090 in., from about 0.025 in. to about 0.070 in., from about 0.030 in. to about 0.060 in., and/or from about 0.040 in. to about 0.050 in. The tubular body 1905 can have an outer diameter OD from about 0.020 in. to about 0.120 in. For example, the outer diameter OD can be from about 0.030 in. to about 0.1 in, from about 0.040 in. to about 0.080 in., from about 0.050 in. to about 0.075 in., and/or from about 0.060 in. to about 0.070 in.

In some cases such as shown in FIG. 9E, a portion of the lumen 1910 proximal to the location of the side port 1960 can have a diameter ID2 smaller than the diameter ID1 of a portion of the lumen 1910 distal to the location of the side port 1960. The diameter ID2 can be from about 0.020 in. to about 0.090 in. For example, the lumen 1910 can have a diameter ID2 from about 0.025 in. to about 0.070 in., from about 0.028 in. to about 0.050 in., and/or from about 0.030 in. to about 0.040 in. The radius R2 of the tubular body 1905 along a portion proximal to the side port 1960 can be from about 0.01 in. to about 0.05 in. For example, the radius R2 can be from about 0.02 in. to about 0.04 in., from about 0.025 in. to about 0.035 in., and/or from about 0.030 in. to about 0.033 in.

The rail 1962 can have a radius R1 from about 0.001 in. to about 0.020 in. For example, the radius R1 can be from about 0.0020 in. to about 0.018 in., from about 0.0040 in. to about 0.016 in., from about 0.0060 in. to about 0.014 in., and/or from about 0.0080 in. to about 0.012 in. When a guidewire is positioned within the rail 1962, the guidewire and the insert catheter 1900 can form a substantially circular cross-section which can create a seal between the insert catheter 1900 and an interior portion of another interventional device. This can beneficially prevent fluid backflow when injecting fluids via the insert catheter 1900.

The insert catheter 1900 can include a distal portion and a proximal portion. The distal portion of the insert catheter 1900 can include the portion of the tubular body 1905 extending from the side port 1960 to the distal end 1905b of the tubular body 1905. The proximal portion of the insert catheter 1900 can include a portion of the tubular body 1905 extending from the side port 1960 to the proximal end 1905a of the tubular body 1905.

In some cases, the tubular body 1905 can include a hypotube. The hypotube can be positioned anywhere along the tubular body 1905. In some cases, the hypotube is positioned on the proximal portion of the insert catheter 1900. For example, at least a portion of the tubular body 1905 extending from the location of the side port 1960 to the proximal end 1905a of the tubular body 1905 can include the hypotube. The hypotube can beneficially prevent buckling when the insert catheter 1900 is positioned and/or translated inside other interventional devices. The hypotube can make the portion of the tubular body 1905 proximal to the side port 1960 stiff enough to prevent buckling or prolapse of the insert catheter 1900 in the aortic arch when one or more interventional devices are being advanced over the insert catheter 1900 and through distal tortuosity and friction but also outside of the body. The stiffness of the more proximal portions of the insert catheter 1900 can allow for easier and faster access to the aortic arch, and also allow the distal end 1905b of the tubular body 1905 to maintain its position relative to an ostium of the aortic arch.

As shown in FIG. 9E, a hypotube 1906 of the tubular body 1905 can have an outer diameter OD2 from about 0.010 in. to about 0.070 in. For example, the outer diameter OD2 can be from about 0.015 in. to about 0.065 in., from about 0.020 in. to about 0.060 in., from about 0.025 in. to about 0.055 in., from about 0.030 in. to about 0.050 in., and/or from about 0.035 in. to about 0.045 in. The hypotube 1906 can extend from a location on the tubular body 1905 proximal to the side port 1960 to the proximal end 1905a of the tubular body 1905. The hypotube 1906 can make the portion of the tubular body 1905 where the hypotube 1906 extends stiffer. This can beneficially provide a better response when the insert catheter is pushed, pulled, and/or rotated (e.g., improved torqueability).

The insert catheter 1900 may be positioned within the vasculature of a patient. For example, the insert catheter 1900 may be tracked up to and positioned within the aortic arch. Once inside the aortic arch, the position and/or orientation of the insert catheter 1900 can be adjusted so that the distal end 1905b of the tubular body 1905 is positioned at the origin of or inside the right brachiocephalic artery, the left common carotid artery (CCA), and/or the left subclavian artery. For example, the distal end 1905b of the tubular body 1905 can be positioned within the right brachiocephalic artery.

A guidewire can be advanced though the lumen 1910 of the insert catheter 1900. As previously described, the guidewire can be inserted advanced/retracted through the lumen 1910 of the tubular body 1905 via the side port 1960. The guidewire can extend beyond the distal end 1905b of the tubular body 1905 into more distal portions of the vasculature. For example, a distal end of the guidewire can be positioned at least as far as the right middle cerebral artery (RMCA) which branches from the right internal carotid artery (RICA), which in turn branches from the right common carotid artery (RCCA).

As previously described herein, one or more catheters may be advanced over the insert catheter 1900 and/or the guidewire. The position of the insert catheter 1900 within the aortic arch can beneficially provide support for the catheters to be advanced into the arteries adjacent to the aortic arch. In some cases, a guide sheath or catheter, which can be similar or identical to the guide sheath 1222 in FIGS. 6A-6F, can be advanced over the insert catheter 1900 and/or the guidewire. The guide catheter can be advanced beyond the distal end 1905b of the tubular body 1905. This can beneficially allow the guide catheter to be positioned into more distal portions of the vasculature.

In some cases, advancing a guidewire through the lumen 1910 of the tubular body 1905 and/or advancing an interventional device over the insert catheter 1900 may alter the shape of the pre-shaped tip 1930. For example, advancing a guidewire through the lumen 1910 extending along the pre-shaped tip 1930 may cause the pre-shaped tip 1930 to lose its original shape and straighten as the guidewire is advanced through the lumen 1910 in the pre-shaped tip 1930. In some cases, when at least a portion of the guidewire is positioned beyond the distal end 1905b of the tubular body 1905, the pre-shaped tip 1930 may assume a substantially straight shape. In such cases, the pre-shaped tip 1930 may extend substantially along a longitudinal axis LA1 of the insert catheter 1900.

The pre-shaped tip 1930 may also lose its original shape and straighten when an interventional device is positioned over the insert catheter 1900. For example, advancing a guide sheath, such as guide sheath 1222 in FIGS. 6A-6F, over the pre-shaped tip 1930 may cause the pre-shaped tip 1930 to lose its original shape and straighten as the guide sheath is advanced through the pre-shaped tip 1930.

Having the pre-shaped tip 1930 straighten can allow the distal end 1905b of the tubular body 1905 to be advanced further into the vasculature of a patient while preventing damage to smaller vessels of the vasculature which the original shape of the pre-shaped tip may cause. In some cases, the pre-shaped tip 1930 may return to its original shape when the guidewire is retracted from the lumen 1910 extending along the pre-shaped tip 1930 and/or when the interventional device is retracted so that the interventional device is no longer positioned over the pre-shaped tip 1930.

Although reference is made to the pre-shaped tip 1930 assuming a straight position when an interventional device is advanced over the pre-shaped tip and/or when a guidewire is extended though the lumen 1910 extending along the pre-shaped tip 1930, the pre-shaped tip 1930 can assume a straight position in other ways. For example, mechanical and/or electrical actuation of the pre-shaped tip 1930 may cause the pre-shaped tip 1930 to transition from having a curved shape (e.g., a stiff configuration) to having a straight shape (e.g., a relaxed configuration), or vice versa.

The pre-shaped tip 1930 may be stiff enough to maintain its shape and/or position within an ostium of the artic arch and allow additional interventional devices to track through (e.g., a guidewire) or over (e.g., first interventional device 2220 and/or a second interventional device 2240) it into supra-aortic vessels, but not so stiff that the curves prevent the insert catheter 1900 from being pushed through other interventional devices into supra-aortic vessels.

The insert catheter 1900 can be used to deliver contrast media, saline, drugs, and/or other types of fluids into the vasculature of a patient (with or without the guidewire in the lumen 1910). In some cases, the insert catheter 1900 can inject fluids, with or without the guidewire in the lumen 1910) at a pressure of up to 1500 PSI. For example, the insert catheter 1900 can inject fluids (with or without the guidewire in the lumen 1910) at a pressure of about 1400 PSI, 1300 PSI, 1200 PSI, 1100 PSI, 900 PSI, 800 PSI. In some cases, the insert catheter 1900 can inject fluids (with or without the guidewire in the lumen 1910) at a flow rate of at least about 1 mL/s. The insert catheter 1900 can inject fluids (with or without the guidewire in the lumen 1910) at a flow rate of about 1 mL/s, 2 mL/s, 3 mL/s, 4 mL/s, 5 mL/s, 8 mL/s, 10 mL/s, 15 mL/s, 20 mL/s, 30 mL/s, 50 mL/s, 100 mL/s, etc.

In some cases, fluid delivery through the insert catheter 1900 can be accomplished using a syringe, such as a syringe 2290, which is described in relation to FIG. 12C. The syringe can be connected to the proximal end of the hub 1950. The syringe may inject fluids at the distal end 1905b of the tubular body 1905 at a flow rate from of at least 0.5 mL/s, 1 mL/s, 2 mL/s, 3 mL/s, 4 mL/s, and/or 5 mL/s. The syringe may accomplish such flow rates even when a guidewire is positioned inside the lumen 1910. When positioned inside another interventional device, such as a first interventional device 2220 and/or a second interventional device 2240, which are described in relation to FIGS. 12A-12C, can encapsulate the side port 1960 of the insert catheter 1900. This can beneficially reduce or prevent leakages at the side port 1960.

FIG. 10 shows an example of a pre-shaped tip for an insert catheter. Any of the insert catheters described herein can include a pre-shaped tip. As shown in FIG. 10, the pre-shaped tip 2030 can include at least one concave side and/or at least one convex side. For example, the pre-shaped tip 2030 can include a first curve defined by a first concave side 2032a and a first convex side 2034a. The first curve can be the proximal most curve. The pre-shaped tip 2030 can include a second curve defined by a second concave side 2032b and a second convex side 2034b. The second curve can be more distal than the first curve. The pre-shaped tip 2030 can include a third curve defined by a third concave side 2032c and a third convex side 2034c. The third curve can be a distal most curve.

The portion of the tubular body 2005 immediately on the proximal side of the first curve (e.g., the longitudinal axis of the tubular body 2005) and the portion of the tubular body 2005 immediately on the distal side of the first curve (e.g., the proximal most portion of the tip 2030) can form an angle A1. The angle A1 can be between about 120° and about 180°. For example, the angle A1 can be between about 130° and about 170°, between about 140° and about 160°, and/or between about 150° and about 160°, and in some cases about 140°, 141°, 142°, 143°, 144°, and/or 145°. The portion of the tubular body 2005 on the proximal side of the first curve can define a longitudinal axis of the insert catheter. The portion of the tubular body 2005 on the distal side of the first curve can be at an oblique takeoff angle from the longitudinal axis of the insert catheter. The first curve can have a radius of curvature from about 15 mm to about 75 mm. For example, the first curve can have a radius of curvature from about 25 mm to about 65 mm, from about 35 mm to about 55 mm, and/or from about 40 mm to about 50 mm.

The second curve results in the tubular body 2005 in the tip 2030 reversing back toward the longitudinal axis. The portions of the tubular body 2005 immediately on the proximal and distal sides of the second curve can establish an angle A2. The angle A2 can be between about 5° and about 65°. For example, the angle A2 can be between about 10° and about 60°, between about 15° and about 55°, between about 20° and about 50°, and/or between about 25° and about 45°. The second curve can have a radius of curvature from about 1 mm to about 20 mm. For example, the second curve can have a radius of curvature from about 5 mm to about 15 mm, from about 7 mm to about 13 mm, and/or from about 8 mm to about 10 mm.

The third curve turns the tubular body 2005 in tip 2030 away from the second curve. The portion of the tubular body 2005 immediately on the distal side of the third curve and the longitudinal axis can establish an angle A3. The angle A3 can be between about 65° and about 125°. For example, the angle A3 can be between about 75° and about 115°, between about 85° and about 105°, and/or between about 90° and about 100°. The third curve can help in the anchorage at the aorta. The third curve can have a radius of curvature from about 5 mm to about 45 mm. For example, the second curve can have a radius of curvature from about 10 mm to about 40 mm, from about 20 mm to about 30 mm, and/or from about 22 mm to about 28 mm.

In some cases, the distance D2 between the distal end 2005b of the tubular body and the portion of the tubular body 2005 where the pre-shaped tip 2030 begins can be between about 25 mm and about 65 mm. For example, the distance D2 can be between about 30 mm and about 60 mm, between about 35 mm and about 55 mm, and/or between about 40 mm and about 50 mm.

The pre-shaped tip 2030 can establish a length L1 between the longitudinal axis LA of the tubular body 2005 and the distal end 2005b. The length L1 can be the shortest distance measured from the longitudinal axis to the distal end 2005b (along an axis perpendicular to the longitudinal axis LA). In some cases, the length L1 can be from about 1 mm to about 30 mm. For example, the length L1 can be from about 5 mm to about 25 mm, from about 10 mm to about 20 mm, and/or from about 12 mm to about 18 mm.

The pre-shaped tip 2030 can establish a length L2 between the longitudinal axis LA of the tubular body 2005 and a lateral-most point of the pre-shaped tip. The lateral-most point of the pre-shaped tip is the farthest location of the pre-shaped tip away from the longitudinal axis of the tubular body 2005. The length L2 can be the shortest distance measured from the longitudinal axis to the lateral-most point of the pre-shaped tip 2030 (along an axis perpendicular to the longitudinal axis LA). In some cases, the length L2 can be from about 5 mm to about 35 mm. For example, the length L2 can be from about 10 mm to about 30 mm, from about 15 mm to about 25 mm, and/or from about 18 mm to about 22 mm.

The pre-shaped tip 2030 can define a length L3 from a point immediately on a distal side of the second curve to the distal end 2005b. The length L3 can be from about 5 mm to about 35 mm. For example, the length L3 can be from about 10 mm to about 30 mm, from about 15 mm to about 25 mm, and/or from about 28 mm to about 22 mm.

FIG. 11 shows another example of a pre-shaped tip for an insert catheter. Any of the insert catheters described herein can include a pre-shaped tip. As shown in FIG. 11, the pre-shaped tip 2130 can include at least one concave side and/or at least one convex side. For example, the pre-shaped tip 2130 can include a first curve defined by a first concave side 2132a and a first convex side 2134a. The first curve can be the proximal most curve. The pre-shaped tip 2130 can include a second curve defined by a second concave side 2132b and a second convex side 2134b. The second curve can be more distal than the first curve. The pre-shaped tip 2130 can include a third curve defined by a third concave side 2132c and a third convex side 2134c. The third curve can be a distal most curve.

The portion of the tubular body 2105 immediately on the proximal side of the first curve (e.g., the longitudinal axis of the tubular body 2005) and the portion of the tubular body 2105 immediately on the distal side of the first curve (e.g., the proximal most portion of the pre-shaped tip 2130) can form an angle A1. The angle A1 can be between about 60° and about 180°. For example, the angle A1 can be between about 70° and about 170°, between about 80° and about 160°, between about 90° and about 150°, between about 100° and about 140°, and/or between about 110° and about 130°, and in some cases about 118°, 119°, 120°, 121°, 122°, and/or 123°. The portion of the tubular body 2105 on the proximal side of the first curve can define a longitudinal axis of the insert catheter. The portion of the tubular body 2005 on the distal side of the first curve can be at an oblique takeoff angle from the longitudinal axis of the insert catheter. The first curve can have a radius of curvature from about 35 mm to about 95 mm. For example, the first curve can have a radius of curvature from about 45 mm to about 85 mm, from about 55 mm to about 75 mm, and/or from about 60 mm to about 70 mm.

The second curve results in the tubular body 2105 in the pre-shaped tip 2130 reversing back toward the longitudinal axis. The portions of the tubular body 2105 immediately on the proximal and distal sides of the second curve can establish an angle A2. The angle A2 can be between about 5° and about 30°. For example, the angle A2 can be between about 10° and about 25, between about 12° and about 23, and/or between about 15° and about 20°. The second curve can have a radius of curvature from about 2 mm to about 15 mm. For example, the second curve can have a radius of curvature from about 5 mm to about 10 mm, and/or from about 6 mm to about 9 mm.

The third curve turns the tubular body 2105 in the pre-shaped tip 2130 away from the second curve. The portion of the tubular body 2105 immediately on the distal side of the third curve and the longitudinal axis can establish an angle A3. The angle A3 can be between about 45° and about 135°. For example, the angle A3 can be between about 55° and about 125°, between about 65° and about 115°, between about 75° and about 105°, between about 80° and about 100°, and/or between about 90° and about 100°. The third curve can help anchor the pre-shaped tip 2130 at the aorta. The third curve can have a radius of curvature from about 5 mm to about 45 mm. For example, the second curve can have a radius of curvature from about 10 mm to about 40 mm, from about 20 mm to about 30 mm, and/or from about 22 mm to about 28 mm.

In some cases, the distance D3 between the distal end 2105b of the tubular body 2105 and the portion of the tubular body 2105 where the pre-shaped tip 2130 begins can be between about 45 mm and about 105 mm. For example, the distance D3 can be between about 55 mm and about 95 mm, between about 65 mm and about 85 mm, and/or between about 70 mm and about 80 mm.

The pre-shaped tip 2130 can establish a length L1 between the longitudinal axis of the tubular body 2105 and the distal end 2105b. The length L1 can be the shortest distance measured from the longitudinal axis to the distal end 2105b. In some cases, the length L1 can be from about 2 mm to about 22 mm. For example, the length L1 can be from about 5 mm to about 20 mm, from about 8 mm to about 17 mm, and/or from about 10 mm to about 15 mm.

The pre-shaped tip 2130 can establish a length L2 between the longitudinal axis of the tubular body 2105 and a lateral-most point of the pre-shaped tip. The lateral most point of the pre-shaped tip is the farthest location of the pre-shaped tip away from the longitudinal axis of the tubular body 2105. The length L2 can be the shortest distance measured from the longitudinal axis to the lateral-most point of the pre-shaped tip 2130. In some cases, the length L2 can be from about 15 mm to about 75 mm. For example, the length L2 can be from about 25 mm to about 65 mm, from about 35 mm to about 55 mm, and/or from about 40 mm to about 50 mm.

The pre-shaped tip 2030 can define a length L3 from a point immediately on a distal side of the second curve to the distal end 2105b. The length L3 can be from about 2 mm to about 22 mm. For example, the length L3 can be from about 5 mm to about 20 mm, from about 8 mm to about 18 mm, and/or from about 10 mm to about 15 mm.

In some implementations, the pre-shaped tip of the insert catheter disclosed herein can have dimensions including but not limited to A1, A2, A3, D1, D2, D3, L1, L2, and/or L3, that range between the corresponding dimensions of the pre-shaped tips in FIGS. 10 and 11.

Although reference is made to the insert catheter 1900 including a pre-shaped tip such as the pre-shaped tip 2030 and/or the pre-shaped tip 2130, which are described in relation to FIGS. 10 and 11, respectively, the insert catheter 1900 can include other pre-shaped tips. Examples of pre-shaped tips that the insert catheter 1900 can have include but are not limited to a VTK shape, a Vert/Berenstein/Davis shape, a SIM shape, and/or a SIM 2 shape.

FIGS. 12A-12C show a stack of interventional devices for conducting a medical procedure. As described herein, the medical procedure can include a procedure for removing a thrombotic occlusion from the vasculature of a patient by applying aspiration via one or more catheters. The stack 2200 of interventional devices can include a first interventional device 2220, a second interventional device 2240, and/or a third interventional device 2260. In some cases, the first interventional device 2220 can include a guide catheter. The first interventional device 2220 can be inserted through an introducer sheath 2210. The introducer sheath 2210 can be introduced at the femoral artery of a patient. In some cases, the introducer sheath 2210 can be introduced at other locations, such as the radial artery and/or the brachial artery. The inner diameter of the introducer sheath 2210 can be larger than an outer diameter of first interventional device 2220 to allow the introducer sheath 2210 to receive the first interventional device 2220. The first interventional device 2220 can include a manifold or hub 2222. The hub 2222 can include or be coupled to a hemostasis valve (e.g., a rotating hemostasis valve; Abbott's COPILOT Bleedback Control Valve, etc.) to accommodate introduction of interventional devices therethrough.

The second interventional device 2240 can include a procedure catheter. The second interventional device 2240 can be inserted through the first interventional device 2220. The inner diameter of the first interventional device 2220 can be larger than an outer diameter of second interventional device 2240 to allow the first interventional device 2220 to receive the second interventional device 2240. The second interventional device 2240 can be introduced into the first interventional device 2220 via the hub 2222. The second interventional device 2240 can include a manifold or hub 2242. The hub 2242 can include or be coupled to a hemostasis valve (e.g., a rotating hemostasis valve) to accommodate introduction of interventional devices therethrough.

The third interventional device 2260 can include an insert catheter. The insert catheter can be similar or identical to any of the insert catheters described herein, including the insert catheters 1300, 1300′, and/or 1900. The third interventional device 2260 can be inserted through the first interventional device 2220 and/or the second interventional device 2240. The inner diameter of the first interventional device 2220 and/or the second interventional device 2240 can be larger than an outer diameter of third interventional device 2260 to allow the first interventional device 2220 and/or the second interventional device 2240 to receive the third interventional device 2260. The third interventional device 2260 can be introduced into the first interventional device 2220 via the hub 2222 and/or into the second interventional device 2240 via the hub 2242. The third interventional device 2260 can include a manifold or hub 2262. The hub 2262 can include or be coupled to a hemostasis valve (e.g., a rotating hemostasis valve) and/or a luer connector to accommodate introduction and/or coupling of interventional devices (e.g., a syringe 2290).

The third interventional device 2260 can include a side port (hidden from view). The side port can be similar or identical to the side ports described in relation to the insert catheters 1300′ and/or 1900. The side port can be in fluid communication with a lumen of the third interventional device 2260. As previously described, the side port can be used to advance and/or retract a guidewire 2270 into the lumen of the third interventional device. As further described below, positioning the side port closer to a distal end of the third interventional device 2260, can beneficially allow clinicians easily and efficiently control all interventional devices during all stages of a procedure.

In some cases, the proximal end 2220a of the first interventional device 2220 and the proximal end 2240a of the second interventional device 2240 can be separated by less than about 65 cm, 55 cm, 45 cm, 35 cm, 25 cm, 20 cm, 15 cm, 10 cm, and, or 5 cm. During a procedure, a maximum distance between the proximal end 2240a of the second interventional device 2240 and the proximal end 2260a of the third interventional device 2260 can be less than about 150 cm, less than about 125 cm, less than about 100 cm, less than about 80 cm, less than about 65 cm, less than about 50 cm, and/or less than about 35 cm. This can ensure that the side port of the third interventional device 2260 stays within the first interventional device 2220 and/or the second interventional device 2240 at all stages of the procedure. Maintaining the side port inside the first interventional device 2220 and/or the second interventional device 2240 at all stages of the procedure can beneficially ensure that fluid being delivered via the third interventional device 2260 does not leak outside the vasculature of a patient via the side port.

When the third interventional device 2260 has been anchored in the desired position along the vasculature of a patient (e.g., the distal end of the third interventional device 2260 is positioned on an ostium of the aortic arch), the first interventional device 2220 and/or the second interventional device 2240 can be advanced further distally into the vasculature of the patient. As the first interventional device 2220 and/or the second interventional device 2240 are advanced, access to the guidewire 2270 will remain available immediately proximal to the hub 2242 of the second interventional device 2240, as shown in FIG. 12B. Thus, at least during one stage of the procedure, a working length can be reduced to the distance D4 between the introducer sheath 2210 and the first portion of the guidewire 2270 positioned outside the stack 2200. The working length can be the maximum distance between the two hands of the clinician in order to handle the stack of interventional devices as shown in FIGS. 12A-12C. In contrast, a third interventional device 2260 without a side port would require the guidewire to be inserted via the proximal end 2260a of the third interventional device 2260. In such cases, the working length would be the distance D5 between the introducer sheath 2210 and the proximal end 2260a of the third interventional device 2260. The distance D4 can range from about 20 cm to about 40 cm, whereas the distance D5 can be from about 60 cm to about 80 cm. In some instances, the distance D4 is within +/− about 5 cm to 10 cm of the clinician's shoulder length, whereas the distance D5 would require the clinician to reach out one or both arms. Reducing the working length can be improve the ergonomics of the stack of interventional devices during an interventional procedure since the distance between the interventional devices that a clinician has to control is reduced to a more comfortable level for the clinician, thus allowing a single clinician to control all the interventional devices in the stack during all stages of the procedure.

As shown in FIG. 12C, all interventional devices in the stack 2200 can be controlled by a single clinician. In some cases, a user's left hand 2202 can control the first interventional device 2220 and/or the introducer sheath 2210. A user's right hand 2204 can control the second interventional device 2240, the third interventional device 2260, and/or the guidewire 2270. The distance between the right hand 2204 and the left hand 2202 are well within typical human ergonomics being only about 15, 14, 13, 12, 11, 10, and/or 9 inches apart, thus allowing manipulation by either hand without having to strain or reach. By having the side port of the third interventional device 2260 positioned inside the first interventional device 2220 and/or the second interventional device 2240, access to the guidewire 2270 can be available proximal to the hub 2222 and/or the hub 2242, even as the first interventional device 2220 and/or the second interventional device 2240 are advanced and/or retracted.

Having the guidewire 2270 follow the proximal end of the hub 2242 can also decrease the total length of the stack 2200. This may beneficially allow for easier handling of the stack 2200 when the stack 2200 is pre-assembled or pre-packaged with all the interventional devices. When the stack 2200 is used in a robotic system, the length of the robotic system may be smaller, more compact, and lighter.

In a stack 2200 without a side port, control of the interventional devices would not be practical and/or ergonomical as a single user would not be able to control all the interventional devices simultaneously. For instance, having the guidewire 2270 extend out of the hub 2262 may require the right hand 2204 to control the guidewire 2270 and/or the third interventional device 2260 and the left hand 2202 to control the first interventional device 2220 and/or the introducer sheath 2210. In such cases, the user would not be able to control the second interventional device 2240, without the assistance of another user. As another example, having the guidewire 2270 extend out of the hub 2262 may require the right hand 2204 to control the guidewire 2270 and the left hand 2202 to control the second interventional device 2240 and/or the third interventional device 2260. In such cases, the user would not be able to control the first interventional device 2220 and/or the introducer sheath 2210, without the assistance of another user.

### Additional Embodiments

Additional embodiments are provided below with reference to FIGS. 1 and 2, but can be implemented in any other example insert catheters disclosed herein. In some embodiments, access for the catheter can be achieved using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. An incision can also be made on right carotid artery or left carotid artery in emergency situations.

Avoiding a tight fit between the guidewire and inside diameter of guidewire lumen enhances the slidability of the catheter over the guidewire. In ultra small diameter catheter designs, it may be desirable to coat the outside surface of the guidewire and/or the inside surface of the wall defining lumen 38 with a lubricous coating to minimize friction as the catheter is axially moved with respect to the guidewire. A variety of coatings may be utilized, such as Parylene, Teflon, silicone, polyimide-polytetrafluoroethylene composite materials or others known in the art and suitable depending upon the material of the guidewire or inner tubular wall 38.

In some embodiments, aspiration catheters which are adapted for intracranial applications generally have a total length in the range of from 60 cm to 250 cm, usually from about 135 cm to about 175 cm. The length of the proximal segment 33 will typically be from 20 cm to 220 cm, more typically from 100 cm to about 120 cm. The length of the distal segment 34 will typically be in the range from 10 cm to about 60 cm, usually from about 25 cm to about 40 cm.

In some examples, the catheters may be composed of any of a variety of biologically compatible polymeric resins having suitable characteristics when formed into the tubular catheter body segments. Exemplary materials include polyvinyl chloride, polyethers, polyamides, polyethylenes, polyurethanes, copolymers thereof, and the like. In one embodiment, both the proximal body segment 33 and distal body segment 34 will comprise a polyvinyl chloride (PVC), with the proximal body segment being formed from a relatively rigid PVC and the distal body segment being formed from a relatively flexible, supple PVC. Optionally, the proximal body segment may be reinforced with a metal or polymeric braid or other conventional reinforcing layer.

The proximal body segment will exhibit sufficient column strength to permit axial positioning of the catheter through a catheter having an inner diameter larger than an outer diameter of the proximal body segment 33 with at least a portion of the proximal body segment 33 extending beyond the larger catheter and into the patient's vasculature. The proximal body segment may have a shore hardness in the range from 50 D to 100 D, often being about 70 D to 80 D. Usually, the proximal shaft will have a flexural modulus from 20,000 psi to 1,000,000 psi, preferably from 100,000 psi to 600,000 psi. The distal body segment 34 will be sufficiently flexible and supple so that it may navigate the patient's more narrow distal vasculature. In highly flexible embodiments, the shore hardness of the distal body segment 34 may be in the range of from about 20 A to about 100 A, and the flexural modulus for the distal segment 34 may be from about 50 psi to about 15,000 psi.

The catheter body may further comprise other components, such as radiopaque fillers; colorants; reinforcing materials; reinforcement layers, such as braids or helical reinforcement elements; or the like. In particular, the proximal body segment may be reinforced in order to enhance its column strength and torqueability (torque transmission) while preferably limiting its wall thickness and outside diameter.

Usually, radiopaque markers will be provided at least at the distal end 14 and the transition region 32 at the distal end of proximal segment 33. One radiopaque marker comprises a metal band which is fully recessed within or carried on the outside of the distal end of the proximal body segment 33. Suitable marker bands can be produced from a variety of materials, including platinum, gold, and tungsten/rhenium alloy. Preferably, the radiopaque marker band will be recessed in an annular channel to produce a smooth exterior surface.

The proximal section 33 of tubular body 16 may be produced in accordance with any of a variety of known techniques for manufacturing interventional catheter bodies, such as by extrusion of appropriate biocompatible polymeric materials. Alternatively, at least a proximal portion or all of the length of tubular body 16 may comprise a polymeric or metal spring coil, solid walled hypodermic needle tubing, or braided reinforced wall, as is known in the microcatheter arts.

In a catheter intended for neurovascular applications, the proximal section 33 of body 16 will typically have an outside diameter within the range of from about 0.117 inches to about 0.078 inches. In one implementation, proximal section 33 has an OD of about 0.104 inches and an ID of about 0.088 inches. The distal section 34 has an OD of about 0.085 inches and an ID of about 0.070 inches.

Diameters outside of the preferred ranges may also be used, provided that the functional consequences of the diameter are acceptable for the intended purpose of the catheter. For example, the lower limit of the diameter for any portion of tubular body 16 in a given application will be a function of the number of fluid or other functional lumen contained in the catheter, together with the acceptable minimum aspiration flow rate and collapse resistance.

Tubular body 16 must have sufficient structural integrity (e.g., column strength or “pushability”) to permit the catheter to be advanced to distal locations without buckling or undesirable bending of the tubular body. The ability of the body 16 to transmit torque may also be desirable, such as to avoid kinking upon rotation, to assist in steering. The tubular body 16, and particularly the distal section 34, may be provided with any of a variety of torque and/or column strength enhancing structures. For example, axially extending stiffening wires, spiral wrapped support layers, braided or woven reinforcement filaments may be built into or layered on the tubular body 16. See, for example, U.S. Pat. No. 5,891,114 to Chien, et al., the disclosure of which is incorporated in its entirety herein by reference.

In many applications, the proximal section 33 will not be required to traverse particularly low profile or tortuous arteries. For example, the proximal section 33 will be mostly or entirely within the relatively large diameter catheter. The transition 32 can be located on the catheter shaft 16 to correspond approximately with or beyond the distal end of the large diameter catheter.

For certain applications, such as intracranial catheterizations, the distal section 34 is preferably at least about 5 cm or 10 cm long and small enough in diameter to pass through vessels as low as 3 mm or 2 mm or lower. The distal section may have a length of at least about 20 cm or 30 cm or 40 cm or more, depending upon the intended target vessel or treatment site.

The distal section, whether carried within the proximal section as an integrated device, or is a separate device to be inserted into the proximal section during a procedure, is substantially shorter than the proximal section. When the distal end of the distal section and the distal end of the proximal section are axially aligned, the proximal end of the distal section is spaced distally apart from the proximal end of the proximal section. The control element such as a control wire or tube spans the distance between the proximal end of the distal section and the proximal manifold or proximal control.

In the foregoing configuration, the proximal end of the distal section will generally be spaced apart distally from the proximal end of the proximal section by at least about 25%, and in some embodiments at least about 50% or 70% or more of the length of the proximal section.

### Disclaimer

Although the present invention has been described in terms of certain preferred embodiments, it may be incorporated into other embodiments by persons of skill in the art in view of the disclosure herein. The scope of the invention is therefore not intended to be limited by the specific embodiments disclosed herein, but is intended to be defined by the full scope of the following claims.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they Can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount in certain embodiments that is within less than plus or minus 10% of, within less than plus or minus 5% of, within less than plus or minus 1% of. within less than plus or minus 0.1% of, and within less than plus or minus 0.01% of the stated amount or characteristic.

1. A system for a vascular procedure, the system comprising:
an introducer sheath comprising a central lumen;
a first interventional device comprising a first lumen and configured to extend along the central lumen of the introducer sheath;
a second interventional device comprising a second lumen and configured to extend along the first lumen of the first interventional device;
a third interventional device configured to extend along the second lumen of the second interventional device, the third interventional device comprising:
an elongate flexible body comprising a proximal end and a distal end, the elongate flexible body having a length of at least about 160 cm,
a third lumen extending from the proximal end to the distal end, and
a side port in fluid communication with the third lumen and positioned along the elongate flexible body, the side port positioned between about 30 cm to about 105 cm from the distal end of the elongate flexible body; and
a guidewire configured to extend through the third lumen and the side port;
wherein, when a distal end of the first interventional device or a distal end of the second interventional device are positioned beyond the distal end of the elongate flexible body of the third interventional device, a distance between the introducer sheath and a portion of the guidewire located immediately proximal to the second interventional device ranges from about 20 cm to about 40 cm.

2. (canceled)

3. The system of claim 1, wherein the elongate flexible body further comprises a pre-shaped tip on a distal portion of the elongate flexible body, and wherein the pre-shaped tip of the elongate flexible body comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 120° to about 180°;
wherein the first section of the pre-shaped tip and a second section of the pre-shaped tip distal to the second curve form an angle from about 5° to about 65°; and
wherein the longitudinal axis of the elongate flexible body and a third section of the pre-shaped tip distal to the third curve form an angle from about 65° to about 125°.

4. The system of claim 3, wherein a distance between the distal end of the elongate flexible body and a longitudinal axis of the elongate flexible body along an axis perpendicular to the longitudinal axis is between about 1 mm and about 30 mm, and wherein a distance between a lateral-most point of the pre-shaped tip and a longitudinal axis of the elongate flexible body along an axis perpendicular to the longitudinal axis is between about 5 mm and about 35 mm.

5. (canceled)

6. The system of claim 1, wherein the elongate flexible body further comprises a pre-shaped tip on a distal portion of the elongate flexible body, and wherein the pre-shaped tip of the elongate flexible body comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 60° to about 180°;
wherein the first section of the pre-shaped tip and a second section of the pre-shaped tip distal to the second curve form an angle from about 5° to about 30°; and
wherein the longitudinal axis of the elongate flexible body and a third section of the pre-shaped tip distal to the third curve form an angle from about 45° to about 135°.

7. The system of claim 6, wherein a distance between the distal end of the elongate flexible body and a longitudinal axis of the elongate flexible body along an axis perpendicular to the longitudinal axis is between about 2 mm and about 22 mm, and wherein a distance between the distal end of the elongate flexible body and a lateral-most point of the pre-shaped tip along an axis perpendicular to a longitudinal axis of the elongate flexible body is between about 15 mm and about 75 mm.

8. (canceled)

9. The system of claim 1, wherein the elongate flexible body further comprises a rail extending from the side port to the proximal end of the elongate flexible body.

10. An insert catheter comprising:
an elongate flexible body comprising a proximal end and a distal end, the elongate flexible body having a length of at least about 160 cm,
a lumen extending from the proximal end to the distal end; and
a side port in fluid communication with the lumen and positioned along the elongate flexible body, the side port positioned between about 30 cm to about 105 cm from the distal end of the elongate flexible body,
wherein the side port is configured to receive a guidewire and place the guidewire in communication with the lumen;
wherein the side port is configured to be positioned within a second interventional device during a vascular procedure.

11. (canceled)

12. The insert catheter of claim 10, further comprising a pre-shaped tip on a distal portion of the elongate flexible body, wherein the pre-shaped tip comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 120° to about 180°;
wherein the first section of the pre-shaped tip and a second section of the pre-shaped tip distal to the second curve form an angle from about 5° to about 65°; and
wherein the second section of the pre-shaped tip and a third section of the pre-shaped tip distal to the third curve form an angle from about 65° to about 125°.

13. The insert catheter of claim 12, wherein a distance between the distal end of the elongate flexible body and a longitudinal axis of the elongate flexible body along an axis perpendicular to the longitudinal axis is between about 1 mm and about 30 mm.

14. The insert catheter of claim 12, wherein a distance between a lateral-most point of the pre-shaped tip and a longitudinal axis of the elongate flexible body along an axis perpendicular to the longitudinal axis is between about 5 mm and about 35 mm.

15. The insert catheter of claim 10, further comprising a pre-shaped tip on a distal portion of the elongate flexible body, wherein the pre-shaped tip of the elongate flexible body comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 60° to about 180°;
wherein the first section of the pre-shaped tip and a second section of the pre-shaped tip distal to the second curve form an angle from about 5° to about 30°; and
wherein the second section of the pre-shaped tip and a third section of the pre-shaped tip distal to the third curve form an angle from about 45° to about 135°.

16. The insert catheter of claim 15, wherein a distance between the distal end of the elongate flexible body and a longitudinal axis of the elongate flexible body along an axis perpendicular to the longitudinal axis is between about 2 mm and about 22 mm.

17. The insert catheter of claim 15, wherein a distance between the distal end of the elongate flexible body and a lateral-most point of the pre-shaped tip along an axis perpendicular to a longitudinal axis of the elongate flexible body is between about 15 mm and about 75 mm.

18. (canceled)

19. A method of gaining and maintaining access to an ostium of the aortic arch using a plurality of interventional devices, the plurality of interventional devices comprising a first interventional device, a second interventional device, and a third interventional device, the method comprising:
placing an introducer sheath into an artery of a patient;
positioning the second interventional device inside the first interventional device;
positioning the third interventional device inside the second interventional device such that a side port of the third interventional device is encapsulated by the second interventional device;
introducing the plurality of interventional devices into a vasculature of the patient via the introducer sheath;
with a distal end of the third interventional device positioned beyond a distal end of the first interventional device and a distal end of the second interventional device, advancing the plurality of interventional devices through the vasculature of the patient until a distal end of the third interventional device is positioned within the aortic arch;
with the distal end of the third interventional device positioned inside the aortic arch, adjusting a position of the distal end of the third interventional device so the distal end of the third interventional device is positioned within an ostium of the aortic arch; and
with the distal end of the third interventional device positioned in the ostium of the aortic arch, advancing at least one of a guidewire, the first interventional device, or the second interventional device beyond the distal end of the third interventional device to get access to supra-aortic vessels;
wherein the side port is positioned between a proximal end of the third interventional device and the distal end of the third interventional device;
wherein a proximal end of the second interventional device comprises a hub;
wherein the guidewire extends through a lumen of the third interventional device;
wherein the guidewire exits the third interventional device via the side port; and
wherein a portion of the guidewire positioned outside of the plurality stack of interventional devices is first available immediately proximal to the hub of the second interventional device.

20. The method of claim 19, wherein a distance between a proximal end of the introducer sheath and the hub of the second interventional device is between about 20 cm to about 40 cm, when the plurality of interventional devices stack is positioned inside the vasculature of the patient.

21. A system for an interventional procedure, the system comprising:
a first interventional device;
a second interventional device comprising a hub and configured to be positioned within the first interventional device;
a third interventional device comprising an elongate body and a hub, wherein the third interventional device is configured to be positioned within the second interventional device, the elongate body comprising,
a proximal end, wherein the hub is coupled to the proximal end of the elongate body,
a distal end,
a lumen extending from the proximal end to the distal end;
a side port positioned on the elongate body between the proximal end and the distal end such that a proximal portion of the elongate body extends from the proximal end to the side port, and a distal portion of the elongate body extends from the distal end to the side port;
wherein the third interventional device is configured to receive a guidewire via the side port; and
wherein when the second interventional device is positioned inside the first interventional device and the third interventional device is positioned inside the second interventional device, the second interventional device, the third interventional device, and the guidewire are configured to be manipulated using a single hand.

22. (canceled)

23. (canceled)

24. The system of interventional devices of claim 21, wherein the side port is positioned between about 30 cm to about 105 cm from the distal end of the third interventional device.

25. The system of interventional devices of claim 21, wherein the side port is positioned inside the second interventional device when the distal end of the third interventional device is positioned beyond a distal end of the first interventional device or a distal end of the second interventional device.

26. The system of interventional devices of claim 21, further comprising an introducer sheath, the introducer sheath configured to receive the first interventional device, the second interventional device, and the third interventional device.

27. The system of interventional devices of claim 26, wherein a distance between the introducer sheath and a portion of the guidewire located immediately proximal to the hub of the second interventional device ranges from about 20 cm to about 40 cm.
